(19) United States
(12) Patent Application Publication
(10) Pub. No.: US 2025/0375418 A1
(43) Pub. Date: Dec. 11, 2025

(54) 1-(NAPHTHALEN-2-YL)-3-AZABICYCLO[3.1.0]HEXANE FOR TREATING MAJOR DEPRESSIVE DISORDER

(71) Applicant: OTSUKA PHARMACEUTICAL CO., LTD., Tokyo (JP)

(72) Inventors: Eva Kohegyi, Langhorne, PA (US); Brian Rothman, Los Angeles, CA (US)

(21) Appl. No.: 19/107,669

(22) PCT Filed: Sep. 1, 2023
(86) PCT No.: PCT/JP2023/032094

§ 371 Date: Feb. 28, 2025

Related U.S. Application Data
(60) Provisional application No. 63/403,624, filed on Sep. 2, 2022.

(51) Int. Cl. A61K 31/403; A61K 9/00; A61K 31/343; A61K 45/06; A61P 25/24

(52) U.S. Cl. CPC A61K 31/403; A61K 9/0004; A61K 31/343; A61K 45/06; A61P 25/24

(57) ABSTRACT

The disclosure relates generally to methods of treating central nervous system disorders using (1R,5S)-1-(naphthalen-2-yl)-3-azabicyclo[3.1.0]hexane (i.e. centanafadine) or a pharmaceutically acceptable salt thereof. More particularly, the disclosure relates to treating conditions affected by monoamine neurotransmitters.

### CROSS-REFERENCE TO RELATED APPLICATIONS

The benefit under 35 U.S.C. § 119(e) of U.S. Provisional Patent Application No. 63/403,624 filed Sep. 2, 2022, is hereby claimed, and the disclosure thereof is hereby incorporated by reference herein.

### TECHNICAL FIELD

### Field of the Disclosure

The disclosure relates generally to methods of treating central nervous system disorders using (1R,5S)-1-(naphthalen-2-yl)-3-azabicyclo[3.1.0]hexane (i.e. centanafadine) or a pharmaceutically acceptable salt thereof. More particularly, the disclosure relates to treating conditions affected by monoamine neurotransmitters.

### Background Art

Major depressive disorder has a severe impact on overall functioning and is a leading cause of disability worldwide in terms of total years lost due to disability. Based on data from 2010, depression-related expenditures in the US were greater than $210 billion, with employers incurring $102 billion in losses due to absenteeism, presenteeism, and disability, and direct medical costs of $99 billion. There is a critical need to improve treatment outcomes associated with major depression.

The American Psychiatric Association recommends healthcare providers to select from various treatment modalities in the acute phase to target remission of a major depressive episode (MDE) and return the patient to the level of functioning prior to the MDE. Currently available options include antidepressant therapy (ADT), depression-focused psychotherapy (e.g., cognitive behavioral therapy), ADT and psychotherapy combination treatment, or other somatic therapies (e.g., electroconvulsive therapy, transcranial magnetic stimulation). Clinical features (e.g., symptom severity, presence of co-occurring disorders or psychosocial stressors) and other factors (e.g., patient preference, prior treatment experiences) influence clinician selection of an initial treatment modality. In most patient populations, when ADT is indicated and psychotherapy is an available and practical option, combining ADT and psychotherapy often provides the quickest and most sustained response. Combination therapy also appears to provide significantly higher improvements in depression symptoms, improvement in quality of life, and increased compliance with treatment.

Additional unmet needs exist amongst those who do receive pharmacotherapy. Current oral therapies that are readily available and prescribed take weeks for effect. The need for rapid treatments has been well-recognized and led to approvals. Patients with routine depression still experience profound disability while awaiting full efficacy during a treatment course.

In addition, some patients may be refractory or resistant to existing ADT treatment and fail to respond to one, or in some cases, multiple monotherapies and combination antidepressant medication treatments. Thus, there remains a need for new methods for treating major depressive disorder.

### SUMMARY OF INVENTION

The disclosure provides a method for treating major depressive disorder in a human patient in need thereof, comprising administering to said patient a therapeutically effective amount of (1R,5S)-1-(naphthalen-2-yl)-3-azabicyclo[3.1.0]hexane, or a pharmaceutically acceptable salt thereof, or a stereoisomer thereof, or a pharmaceutically acceptable salt of a stereoisomer thereof.

In a further embodiment, the disclosure provides a method for treating major depressive disorder in a human patient in need thereof, comprising administering to said patient a therapeutically effective amount of (1R,5S)-1-(naphthalen-2-yl)-3-azabicyclo[3.1.0]hexane, or a pharmaceutically acceptable salt thereof, or a stereoisomer thereof, or a pharmaceutically acceptable salt of a stereoisomer thereof, in combination with a one or more antidepressants, optionally selected from selective serotonin reuptake inhibitors (SSRIs) and/or serotonin-norepinephrine reuptake inhibitors (SNRIs).

In a further embodiment, the disclosure provides a method for treating major depressive disorder in a human patient in need thereof, comprising administering to said patient a therapeutically effective amount of (1R,5S)-1-(naphthalen-2-yl)-3-azabicyclo[3.1.0]hexane, or a pharmaceutically acceptable salt thereof, or a stereoisomer thereof, or a pharmaceutically acceptable salt of a stereoisomer thereof, in combination with one selective serotonin reuptake inhibitor (SSRIs).

Provided herein are methods for treating major depressive disorder in a human patient in need thereof, comprising administering to said patient a therapeutically effective amount of centanafadine or a pharmaceutically acceptable salt thereof, to effectively treat MDD, wherein centanafadine is optionally administered in an extended-release dosage form comprising a plurality of CTN beads, the plurality of centanafadine beads each comprising a core particle comprising centanafadine or a pharmaceutically acceptable salt thereof, and an excipient, and wherein the plurality of centanafadine beads comprises a mixture of one or more immediate release (IR) beads, one or more sustained release (SR) beads, and one or more delayed release (DR) beads.

Centanafadine, a molecular entity with inhibitory activity at the norepinephrine (NE), dopamine (DA), and serotonin (5-HT)-reuptake transporters, has been shown to be generally safe and well tolerated, as indicated by a low rate of adverse events (AEs).

Provided herein are methods for treating major depressive disorder in a patient in need thereof which comprises administering to said patient centanafadine extended-release capsules as monotherapy. Provided herein are methods for treating major depressive disorder in a patient in need thereof which comprises administering to said patient centanafadine extended-release capsules as adjunct to selective serotonin reuptake inhibitor(s).

Provided herein are methods for alleviating major depressive disorder symptoms in a patient in need thereof, which comprises administering to said patient centanafadine extended-release capsules as monotherapy. Provided herein are methods for alleviating major depressive disorder symptoms, in a patient in need thereof which comprises administering to said patient centanafadine extended-release capsules as adjunct to selective serotonin reuptake inhibitor(s).

Also provided herein are methods for treating major depressive disorder in a patient in need thereof, which comprise administering to said patient, a) a therapeutically effective amount of (1R,5S)-1-(naphthalen-2-yl)-3-azabicyclo[3.1.0]hexane (CTN) or a pharmaceutically acceptable salt thereof, and b) one or more antidepressants selected from selective serotonin reuptake inhibitors (SSRIs), wherein the SSRI is selected from fluoxetine, sertraline, paroxetine, citalopram, escitalopram, fluvoxamine, vilazodone, venlafaxine, desvenlafaxine, dapoxetine, vortioxetine, venlafaxine and duloxetine; and wherein CTN is optionally administered in an extended-release dosage form comprising a plurality of CTN beads, the plurality of CTN beads each comprising a core particle comprising CTN or a pharmaceutically acceptable salt thereof, and an excipient, and wherein the plurality of CTN beads comprises a mixture of one or more immediate release (IR) beads, one or more sustained release (SR) beads, and one or more delayed release (DR) beads.

Also provided herein are methods for treating major depressive disorder in a patient in need thereof, which comprise administering to said patient, a) a therapeutically effective amount of (1R,5S)-1-(naphthalen-2-yl)-3-azabicyclo[3.1.0]hexane (CTN) or a pharmaceutically acceptable salt thereof, and b) one or more antidepressants selected from serotonin-norepinephrine reuptake inhibitors (SNRIs), wherein CTN is optionally administered in an extended-release dosage form comprising a plurality of CTN beads, the plurality of CTN beads each comprising a core particle comprising CTN or a pharmaceutically acceptable salt thereof, and an excipient, and wherein the plurality of CTN beads comprises a mixture of one or more immediate release (IR) beads, one or more sustained release (SR) beads, and one or more delayed release (DR) beads.

Methods for treating major depressive disorder (MDD) in a patient in need thereof may comprise administering to said patient, (1R,5S)-1-(naphthalen-2-yl)-3-azabicyclo[3.1.0]hexane (CTN) or a pharmaceutically acceptable salt thereof, in combination with other classes of antidepressants such as, but not limited to, benzodiazepines, anxiolytics, noradrenergic and specific serotonergic antidepressants (NaSSAs), tricyclic antidepressants (TCAs) and monoamine oxidase inhibitors (MAOIs).

The disclosure further provides (1R,5S)-1-(naphthalen-2-yl)-3-aza-bicyclo[3.1.0]hexane or a pharmaceutically acceptable salt thereof for use in the methods disclosed herein.

For the compositions, methods, and uses described herein, optional features, including but not limited to components, compositional ranges thereof, substituents, conditions, and steps, are contemplated to be selected from the various aspects, embodiments, and examples provided herein.

Further aspects and advantages will be apparent to those of ordinary skill in the art from a review of the following detailed description, taken in conjunction with the drawings. While the compositions, methods, and uses described herein are susceptible of embodiments in various forms, the description hereafter includes specific embodiments with the understanding that the disclosure is illustrative and is not intended to limit the invention to the specific embodiments described herein.

### DESCRIPTION OF EMBODIMENTS

Described herein are methods of using centanafadine (CTN) or a pharmaceutically acceptable salt thereof for the treatment of central nervous system disorders. CTN is classified as a BSC Class I molecule, which is highly soluble and highly permeable. As used herein, CTN should be understood to refer to centanafadine, while pharmaceutically acceptable salts thereof are also considered in addition to or as one or more alternatives to CTN in every method, use, and formulation described herein, unless explicitly stated otherwise. Centanafadine [CAS 924012-43-1], also known chemically as (1R,5S)-1-(naphthalen-2-yl)-3-azabicyclo[3.1.0]hexane or (+)-1-(naphthalen-2-yl)-3-azabicyclo[3.1.0]hexane, has the following structure:

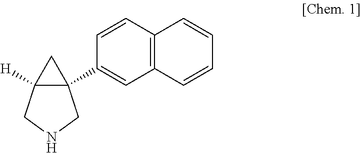 [SMILES: c1ccc2cc([C@]34CNC[C@H]3C4)ccc2c1]

and is an unbalanced triple reuptake inhibitor with the most potency towards the norepinephrine reuptake transporter (NET), one-sixth as much towards the dopamine reuptake transporter (DAT), and one-fourteenth as much towards the serotonin reuptake transporter (SERT).

The disclosure provides methods for treating major depressive disorder comprising administering a therapeutically effective amount of centanafadine or a pharmaceutically acceptable salt, wherein the centanafadine optionally is administered in an extended-release dosage form comprising a plurality of beads, as described herein. In some embodiments, the disclosure provides centanafadine or a pharmaceutically acceptable salt thereof for use in the disclosed methods.

The terms “centanafadine”, “(1R,5S)-1-(naphthalen-2-yl)-3-azabicyclo[3.1.0]hexane”, “(+)-1-(naphthalen-2-yl)-3-azabicyclo[3.1.0]hexane”, and “CTN” are used interchangeably herein. The term “centanafadine” as used herein includes “(1R,5S)-1-(naphthalen-2-yl)-3-azabicyclo[3.1.0]hexane” optionally containing less than 5, 3, 2, 1, 0.5 or 0.2% by weight of “(1S,5R)-1-(naphthalen-2-yl)-3-azabicyclo[3.1.0]hexane”, based on a 100% total weight of compound.

The terms “(1S,5R)-1-(naphthalen-2-yl)-3-azabicyclo[3.1.0]hexane” and “(−)-1-(naphthalen-2-yl)-3-azabicyclo[3.1.0]hexane”, which refer to the enantiomer of (−)-enantiomer of CTN, are used interchangeably herein.

In some embodiments described herein comprising administering centanafadine or a pharmaceutically acceptable salt thereof, centanafadine may comprise different amounts of other stereoisomers.

In any embodiment comprising administering CTN or a pharmaceutically acceptable salt thereof, it is contemplated that the CTN is substantially free of (1S,5R)-1-(naphthalen-2-yl)-3-azabicyclo[3.1.0]hexane.

As used herein, “substantially free of (1S,5R)-1-(naphthalen-2-yl)-3-aza-bicyclo[3.1.0]hexane” or “substantially free of the corresponding (−) enantiomer” means at least more of (1R,5S)-1-(naphthalen-2-yl)-3-azabicyclo[3.1.0]hexane than the corresponding (−) enantiomer. Optionally, “substantially free of the corresponding (−) enantiomer” means containing no more than 5% w/w (weight/weight) of the corresponding (−) enantiomer, in free or pharmaceutically acceptable salt form, optionally no more than 3% w/w of the corresponding (−) enantiomer, in free or pharmaceutically acceptable salt form, optionally no more than 2% w/w of the corresponding (−) enantiomer, in free or pharmaceutically acceptable salt form, optionally no more than 1% w/w of the corresponding (−) enantiomer, in free or pharmaceutically acceptable salt form, optionally no more than 0.5% w/w of the corresponding (−) enantiomer, in free or pharmaceutically acceptable salt form, optionally no more than 0.2% w/w of the corresponding (−) enantiomer, in free or pharmaceutically acceptable salt form.

As used herein, any aspect or embodiment comprising “(1R,5S)-1-(naphthalen-2-yl)-3-azabicyclo[3.1.0]hexane” is contemplated to include or use the compound in any form, for example, free base or a pharmaceutically acceptable salt form, e.g., as a pharmaceutically acceptable acid addition salt.

The term “pharmaceutically acceptable” generally means biologically or pharmacologically compatible for in vivo use in animals or humans, and optionally means approved by a regulatory agency of the Federal or a state government or listed in the U.S. Pharmacopoeia or other generally recognized pharmacopoeia for use in animals, and more particularly in humans.

Pharmaceutically acceptable salts are known in the art and include salts that are physiologically acceptable at the dosage amount and form to be administered. CTN is provided as a CTN hydrochloride salt.

As used herein, any aspect or embodiment comprising “(1R,5S)-1-(naphthalen-2-yl)-3-azabicyclo[3.1.0]hexane” is also contemplated to include or use the compound in crystalline or amorphous form including, for example, polymorphs, solvates (including hydrates), unsolvated polymorphs (including anhydrates), conformational polymorphs, and amorphous forms of the compounds, as well as mixtures thereof. “Crystalline form” and “polymorph” may be used interchangeably herein, and are meant to encompass any crystalline forms of (1R,5S)-1-(naphthalen-2-yl)-3-azabicyclo[3.1.0]hexane, in free or pharmaceutically acceptable salt form, including, for example, polymorphs, solvates (including hydrates), unsolvated polymorphs (including anhydrates), and conformational polymorphs, as well as mixtures thereof, unless a particular crystalline form is referred to.

In the description herein, the amounts of CTN, weight percentages of CTN, or ranges thereof in the pharmaceutical formulations (e.g., tablets, capsules, beads, core particles, etc.), and in any method or use disclosed herein, are applicable to CTN free base, as well as to pharmaceutically acceptable salts thereof, such that any description by weight should be viewed as for CTN free base, and in addition in the alternative as description applicable to a pharmaceutically acceptable salt form, unless specified otherwise. In each such description, CTN as the hydrochloride salt is specifically contemplated.

Crystalline and amorphous forms of (1R,5S)-1-(naphthalen-2-yl)-3-azabicyclo[3.1.0]hexane may be used in any combination or in forms that are substantially free of one or more of the other crystalline forms or free of the amorphous form.

As used herein, “substantially free of other polymorphic forms” means that the crystalline material contains no more than 20% of any other crystalline form, optionally 10% w/w of any other crystalline form, optionally no more than 5% w/w of any other crystalline form, or no more than 2% w/w of any other crystalline form, optionally no more than 1% w/w of any other crystalline form.

(1R,5S)-1-(Naphthalen-2-yl)-3-azabicyclo[3.1.0]hexane can be provided or used in prodrug form. Prodrugs are considered to be any covalently bonded carriers that release the active parent drug in vivo.

(1R,5S)-1-(Naphthalen-2-yl)-3-azabicyclo[3.1.0]hexane can be synthesized as described in U.S. Pat. No. 8,461,196, International Publication Nos. WO2007/016155, WO 2013/019271 and WO 2021/075494, or Japanese Patent JP 2019147794A, each of which is incorporated herein by reference in their entirety.

As used herein, the term “escitalopram”, chemical name (S)-1-[3-(dimethyl amino)propyl]-1-(4-fluorophenyl)-1,3-dihydroisobenzofuran-5-carbonitrile, is the S-enantiomer of citalopram. Escitalopram is a selective serotonin reuptake inhibitor. The term “escitalopram” also includes polymorphs, hydrates, solvates, and amorphous forms of escitalopram and its pharmaceutically acceptable salts. Escitalopram oxalate is currently marketed in the United States as Lexapro™ for the treatment of major depressive disorder and generalized anxiety disorder.

As used herein, the term “patient” includes human or non-human (i.e., animal) patient. In some embodiments, patient encompasses both human and nonhuman. In some embodiments, patient means a nonhuman. In other embodiments, patient means a human.

As used herein, “therapeutically effective amount” or “therapeutic dose” refers to an amount effective, when administered to a human or non-human patient, to provide a therapeutic benefit, optionally one or more of prevention of symptoms, amelioration of symptoms, and delay in the development, of symptoms. Depressive symptoms include low mood, diminished interest in activities, psychomotor slowing or agitation, changes in appetite, poor concentration or indecisiveness, excessive guilt or feelings of worthlessness, and suicidal ideations may occur in the context of depressive disorders, bipolar disorders, mood disorders due to a general medical condition, substance-induced mood disorders, other unspecified mood disorders, and also may be present in association with a range of other psychiatric disorders, including but not limited to psychotic disorders, cognitive disorders, eating disorders, anxiety disorders and personality disorders.

The specific dose of substance administered to obtain a therapeutic benefit will, of course, be determined by the particular circumstances surrounding the case, including, for example, the specific substance administered, the route of administration, the condition being treated, and the individual being treated.

As used herein, the terms “improvements in depression symptoms” refers to a gradual decrease in one or more depression symptoms selected from avoidance, isolation, withdrawal, anxiety, suicidality; and increase engagement in activities which were enjoyed previously; and increase in activities that have been shown to improve mood.

As used herein, the term “alleviating” refers to making less severe, lightening, mitigating, relieving, ease, easier to deal with.

As used herein, the term “relieving” refers to making less severe, lightening, mitigating, alleviating, ease, easier to deal with.

As used herein, the term “concurrently” means the compounds are administered “simultaneously”, at the same time or within the same composition. In embodiments, the compounds can be administered simultaneously. In embodiments, the compounds can be administered within the same composition.

As used herein, the term “sequentially” means the compounds are administered in a certain order or consecutively.

In one type of embodiment, the centanafadine and escitalopram may be co-administered or administered in a unitary oral dosage form containing both actives. In another related embodiment, centanafadine and escitalopram may be co-administered or administered in two or more oral dosage forms containing one or both actives. Optionally, the unitary dosage form or separate dosage forms are once daily formulations, i.e., one administration of each dosage form daily is sufficient to treat the desired CNS disorder. In still another embodiment, centanafadine and escitalopram may be administered sequentially.

In still another embodiment, centanafadine and escitalopram may be administered at different times of the day, for instance centanafadine may be administered during the morning and escitalopram may be administered during the evening. In still another embodiment, centanafadine and escitalopram may be administered with food. In still another embodiment, centanafadine and escitalopram may be administered without food. In still another embodiment, centanafadine may be administered with food and escitalopram may be administered without food. Alternatively, in another embodiment, centanafadine may be administered without food and escitalopram may be administered with food.

If a patient needs to discontinue treatment with centanafadine and escitalopram, in order to minimize symptoms of withdrawal, the patient can slowly taper off the medication by slowly decreasing the dose of medication(s) until the body adjusts to the lower levels of medications(s).

As used herein, the term “comprising” indicates the potential inclusion of other agents, elements, steps, or features, in addition to those specified.

As used herein, the term “treat” includes one or more of the following:
(a) relieving or alleviating at least one symptom of major depression disorder in a subject,
(b) relieving or alleviating the intensity and/or duration of a manifestation of major depression disorder experienced by a subject, and
(c) arresting, delaying the onset (i.e., the period prior to clinical manifestation of major depression disorder) and/or reducing the risk of developing or worsening of major depression disorder.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

Patients suffering from “treatment resistant depression” include (1) those who fail to respond to standard doses (i.e., significantly superior to placebo in double-blind studies) of antidepressants (such as SSRIs) administered continuously for a minimum duration of 6 weeks, and (2) those who fail to respond to standard doses of an antidepressant (such as an SSRI) (monotherapy) administered continuously for a minimum duration of 12 weeks.

As used herein, the term “extended-release dosage form” is equivalent to prolonged release. A dosage form can be characterized by its overall release profile, e.g. the profile resulting from multiple regions when present in the dosage form. A dosage form exhibiting extended-release characteristics can be characterized as a sustained release dosage form, even if it further contains an immediate release region in addition to a sustained release formulation region, e.g. bead. Similarly, a dosage form exhibiting extended-release characteristics can be characterized as a sustained release dosage form, even if it further contains a delayed release region in addition to a sustained release formulation region, e.g. bead.

The terms “extended-release”, “sustained release”, “modified release”, and “sustained or modified release” as used herein refer to the release of an active ingredient over an extended period of time leading to lower peak plasma concentrations and a prolonged T max as compared to immediate release formulations.

As used herein “XR” refers to extended release.

As used herein “QD” refers to “once a day”.

As used herein “as adjunct” refers to another treatment used together with the primary treatment, with the purpose to assist the primary treatment, or in combination or co-administered with the primary treatment. For example, the primary treatment with centanafadine or a pharmaceutically acceptable salt thereof can be co-administered with a secondary treatment, or administered on other schedules during a course of treatment.

As used herein, the term “co-administer” refers to administration of two or more agents within a 24-hour period of each other, for example, as part of a clinical treatment regimen. Optionally, “co-administer” refers to administration of two or more agents within 2 hours of each other. Optionally, “co-administer” refers to administration of two or more agents within 30 minutes of each other. Optionally, “co-administer” refers to administration of two or more agents within 15 minutes of each other. Optionally, “co-administer” refers to administration at the same time, either as part of a single formulation or as multiple formulations that are administered by the same or different routes.

As used herein, an adverse event (AE) refers to any untoward medical occurrence in a patient which does not necessarily have a causal relationship with the disclosed treatments. Adverse events do not include information recorded as medical history at screening for pre-planned procedures for which the underlying condition was known and no worsening occurred.

An adverse reaction is any untoward and unintended response to an investigational medical product (IMP) of any dose administered. AEs are graded on a 3-point scale, wherein 1 is mild (e.g., discomfort is noticed, but no disruption to daily activity); 2 is moderate (e.g., discomfort is sufficient to reduce or affect normal daily activity); and 3 is severe (e.g., inability to work or perform normal daily activity).

A suspected adverse reaction is any AE for which there is a reasonable possibility that the IMP caused the AE. For the purpose of Investigational New Drug (IND) safety reporting, “reasonable possibility” means there is evidence to suggest a causal relationship between the IMP and the AE. Suspected adverse reaction implies a lesser degree of certainty about causality.

As used herein, the term “treatment-emergent AEs” (TEAEs) are defined as AEs with an onset date on or after the start of double-blind treatment. In more detail, TEAEs are all AEs which started after the start of double-blind IMP treatment; or if the event was continuous from baseline and was worsening, serious, IMP-related, or resulted in death, discontinuation, interruption, or reduction of IMP.

As used herein, the term “serious adverse event” (SAE) includes any event that results in any of the following outcomes: death; life-threatening, i.e., the subject was, at immediate risk of death from the event as it occurred, not including an event that, had it occurred in a more severe form, might have caused death; persistent or significant incapacity/disability or substantial disruption of the ability to conduct normal life functions; requires inpatient hospitalization or prolongs hospitalization; congenital anomaly/birth defect; other medically significant events that, based upon appropriate medical judgment, may jeopardize a subject and may require medical or surgical intervention to prevent one of the outcomes listed above; e.g., allergic bronchospasm requiring intensive treatment in an emergency room or home, blood dyscrasias or convulsions that do not result in hospitalization, or the development of drug dependency or drug abuse.

As used herein, a “nonserious adverse event” are all AEs that do not meet the criteria for a “serious” AE. As used herein, “adverse events of special interest” refers to a noteworthy event for the particular product/IMP or class of products that a sponsor may wish to monitor carefully.

### Extended-Release Centanafadine (CTN XR) Dosage Forms

The methods disclosed herein can include administering CTN or a pharmaceutically acceptable salt thereof in an extended-release dosage form comprising a plurality of CTN beads, the plurality of CTN beads each comprising a core particle comprising CTN or a pharmaceutically acceptable salt thereof, and an excipient, and wherein the plurality of CTN beads comprises a mixture of one or more immediate release (IR) beads, one or more sustained release (SR) beads, and one or more delayed release (DR) beads. In the description below, CTN beads are described as an example of such formulation types. The characteristics for the formulations described, e.g. ratios of different bead types, are also applicable to unit dosage forms, e.g. collections of beads disposed in capsules.

Suitable extended-release CTN dosage forms are described in U.S. patent application Ser. No. 17/677,726 filed on Feb. 22, 2022, the disclosure of which is incorporated herein by reference in its entirety.

The disclosed dosage forms can be measured for the appropriate CTN dosage strength prior to administration, or the formulation can be packaged in unit dosage form, e.g. in capsules, sachets, etc. In the alternative, the formulation can be compounded in unit dosage form, e.g. by pressing into a monolithic unit form, such as a tablet.

The extended-release dosage forms can include a plurality of CTN-containing regions, and optionally the plurality of CTN regions can include one or more of release characteristics, e.g. selected from delayed release, sustained release, immediate release, and delayed-sustained release. The regions can be physically joined or separated. For example, one type of dosage form includes a plurality of CTN-containing beads (CTN beads), the plurality of CTN beads each including a core particle and an excipient. The plurality of CTN beads can include one or more of types of beads selected from an immediate release bead, a sustained release bead, and a delayed release bead, and optionally a combination of one or more immediate release beads, one or more sustained release beads, and one or more delayed release beads.

The extended-release dosage forms can be provided in unit dose form, for example as a collection of beads disposed in a capsule shell, or as a collection of beads disposed in a sachet. In another type of embodiment, a collection of granules is pressed into tablet form, with or without extragranular components, e.g. an extragranular disintegrant. Other forms will be evident to the skilled artisan in view of the disclosure herein.

Also provided for the methods and uses described herein is a dosage form comprising CTN and an excipient, wherein the pharmaceutical dosage form has an in vivo absorption profile that is bimodal.

As mentioned above, a dosage form according to the disclosure can include a plurality of CTN beads, including one or more types selected from: an immediate release bead, a sustained release bead, a delayed release bead, and a delayed-sustained release bead. The plurality of CTN-containing beads can include at least a portion of the beads including a delayed release or delayed-sustained release coating, at least a portion of the beads including a sustained release coating, and at least a portion of the beads being immediate release beads. Such a dosage form has been shown to exhibit advantageous pharmacokinetics, to be suitable for administration to pediatric subjects, and suitable for administration once daily. Without intending to be bound by any particular theory, it is contemplated that the pharmacokinetics are influenced by the plurality of CTN beads having a combination of bead types, including immediate release, sustained release, and delayed release.

As used herein, the term “wt. %” is the weight percent based on the total weight of the thing described, e.g. of the core particle, or coating, or total bead, as described in context or explicitly. Unless described otherwise, the “wt. %” is intended to describe the weight percent based on dry weight (e.g., for a core particle following drying). Unless described otherwise, the terms “wt. %” and “% by weight” are used interchangeably herein.

While the description herein refers to beads, and beads such as those made by extrusion and spheronization can have certain advantages such as greater uniformity and size, particles of any size and shape and made by other processes are equally contemplated as alternatives, as are monolithic dosage forms.

All ranges set forth herein include all possible subsets of ranges and any combinations of such subset ranges. By default, ranges are inclusive of the stated endpoints, unless stated otherwise. Where a range of values is provided, it is understood that each intervening value between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also contemplated to be part of the disclosure.

### Bead Formulation

In some embodiments, the plurality of beads includes a mixture of one or more immediate release beads, and one or more sustained release beads. In some embodiments, the amount of CTN by weight can be present at a ratio in a range of about 1:100 to about 1:1 in the collection of one or more immediate release beads to the collection of one or more sustained release beads. In some embodiments, the amount of CTN by weight can be present at a ratio in a range of about 1:50 to about 1:1 in the collection of one or more immediate release beads to the collection of one or more sustained release beads, or in a range of about 1:20 to about 1:1, or in a range of about 1:15 to about 1:1, for example, 1:10.

In some embodiments, the plurality of beads includes a mixture of one or more immediate release beads and one or more delayed release beads. In some embodiments, the amount of CTN by weight can be present at a ratio in a range of about 1:100 to about 1:1 in the collection of one or more immediate release beads to the collection of one or more delayed release beads. In some embodiments, the amount of CTN by weight can be present at a ratio in a range of about 1:50 to about 1:1 in the collection of one or more immediate release beads to the collection of one or more delayed release beads, or in a range of about 1:20 to about 1:1, or in a range of about 1:15 to about 1:1, for example, 1:10.

In some embodiments, the plurality of beads includes a mixture of one or more delayed release beads and one or more sustained release beads. In some embodiments, the amount of CTN by weight can be present at a ratio in a range of about 5:1 to about 1:5 in the collection of one or more delayed release beads to the collection of one or more sustained release beads, in a range of about 3:1 to about 1:3, or in a range of about 2:1 to about 1:2, or in a range of about 1.5:1 to about 1:1.5, for example about 1:1.

In some embodiments, the plurality of beads includes a mixture of one or more immediate release beads and one or more delayed-sustained release beads. In some embodiments, the amount of CTN by weight can be present at a ratio in a range of about 1:100 to about 1:1 in the collection of one or more immediate release beads to the collection of one or more delayed-sustained release beads. In some embodiments, the amount of CTN by weight can be present at a ratio in a range of about 1:50 to about 1:1 in the collection of one or more immediate release beads to the collection of one or more delayed-sustained release beads, or in a range of about 1:20 to about 1:1, or in a range of about 1:15 to about 1:1, for example, 1:10.

In some embodiments, the plurality of beads includes a mixture of one or more immediate release beads, one or more sustained release beads, and one or more delayed release beads. In embodiments, the ratio of CTN can be present in the one or more immediate release beads, one or more sustained release beads, and one or more delayed release beads at a ratio in a range of about 0.1-1:1-20:1-20 parts by weight based on the weight of CTN. In some embodiments, the ratio of CTN can be present in the one or more immediate release beads, one or more sustained release beads, and one or more delayed release beads at a ratio in a range of about 0.5-1:5-20:5-20 parts by weight based on the weight of CTN. In some embodiments, the ratio of CTN or pharmaceutically acceptable salt thereof can be present in the one or more immediate release beads, one or more sustained release beads, and one or more delayed release beads at a ratio in a range of about 0.7-1.3:3-6:3-6 parts by weight based on the weight of CTN. In embodiments, the ratio of CTN or pharmaceutically acceptable salt thereof can be present in the one or more immediate release beads, one or more sustained release beads, and one or more delayed release beads at a ratio in a range of about 0.7-1:5-15:5-15 parts by weight based on the weight of CTN. For example, the ratio of CTN or pharmaceutically acceptable salt thereof can be present in the one or more immediate release beads, one or more sustained release beads, and one or more delayed release beads can be 1:3.6:3.6.

In some embodiments, the plurality of beads comprises a mixture of one or more immediate release beads, one or more sustained release beads, and one or more delayed-sustained release beads. In some embodiments, the ratio of CTN can be present in the one or more immediate release beads, one or more sustained release beads, and one or more delayed-sustained release beads at a ratio in a range of about 0.1-1:1-20:1-20 parts by weight based on the weight of CTN. In some embodiments, the ratio of CTN or pharmaceutically acceptable salt thereof can be present in the one or more immediate release beads, one or more sustained release beads, and one or more delayed-sustained release beads at a ratio in a range of about 0.5-1:5-20:5-20 parts by weight based on the weight of CTN. In some embodiments, the ratio of CTN or pharmaceutically acceptable salt thereof can be present in the one or more immediate release beads, one or more sustained release beads, and one or more delayed-sustained release beads at a ratio in a range of about 0.7-1.3:3-6:3-6 parts by weight based on the weight of CTN. In some embodiments, the ratio of CTN or pharmaceutically acceptable salt thereof can be present in the one or more immediate release beads, one or more sustained release beads, and one or more delayed-sustained release beads at a ratio in a range of about 0.7-1:5-15:5-15 parts by weight based on the weight of CTN. For example, the ratio of CTN or pharmaceutically acceptable salt thereof can be present in the one or more immediate release beads, one or more sustained release beads, and one or more delayed-sustained release beads at a ratio of about 1:3.6:3.6 parts by weight based on the weight of CTN.

In some embodiments, the immediate release beads can be present in the formulation or dosage form in an amount in a range of about 1% to about 75% based on the total weight of the plurality of CTN beads in the formulation or dosage form. For example, the immediate release beads are present in the dosage form, in an amount in a range of about 1% to about 60%, or about 1% to about 50%, or about 5% to about 50%, or about 5% to about 40%, or about 5% to about 30%, or about 5% to about 25%, or about 10% to about 30%, 9% to about 55% or about 18% to about 28%, or about 5% to about 20%, or about 5% to about 15%, or about 1% to about 25%, or about 1% to about 10%, based on the total weight of the plurality of CTN beads. In embodiments, the immediate release beads are present in the dosage form in an amount in a range of about 1% to about 50% based on the total weight of the plurality of CTN beads. In embodiments, the immediate release beads are present in the dosage form in an amount in a range of about 1% to about 25% based on the total weight of the plurality of CTN beads. In embodiments, the immediate release beads are present in the dosage form in an amount in a range of about 1% to about 10% based on the total weight of the plurality of CTN beads. In some embodiments, the immediate release beads can be present in the dosage form in an amount in a range of about 9% to about 19% based on the total weight of the plurality of CTN beads; such embodiments are particularly contemplated when the drug loading in the bead is about 40 wt. % to about 60 wt. %, e.g. 50 wt. %. In some embodiments, the immediate release beads can be present in the dosage form in an amount in a range of about 40% to about 55% based on the total weight of the plurality of CTN beads; such embodiments are particularly contemplated when the drug loading in the bead is about 5 wt. % to about 15 wt. %, e.g. 10 wt. %. In some embodiments, the immediate release beads are present in the dosage form in an amount in a range of about 18% to about 28% based on the total weight of the plurality of CTN beads.

In some embodiments, the immediate release bead, or a bead core, can include CTN in an amount in a range of about 5 wt. % to about 90 wt. % based on the total weight of the bead or bead core. For example, the immediate release bead or a bead core can include CTN in an amount in a range of about 5 wt. % to about 85 wt. %, about 5 wt. % to about 80 wt. %, about 5 wt. % to about 60 wt. %, about 5 wt. % to about 30 wt. %, or about 25 wt. % to about 60 wt. %, or about 40 wt. % to about 60 wt. %, or about 5 wt. % to about 15 wt. %, based on the total weight of the bead or bead core. In some embodiments, the immediate release bead or a bead core can include CTN in an amount in a range of 5 wt. % to 15 wt. % based on the total weight of the bead or bead core. In some embodiments, the immediate release bead or a bead core can include CTN in an amount in a range of 40 wt. % to 60 wt. % in the immediate release bead based on the total weight of the immediate release bead or bead core. In some embodiments, the immediate release bead or a bead core can include the CTN in an amount in a range of about 70 wt. % to about 90 wt. %, or about 75 wt. % to about 80 wt. % based on the total weight of the immediate release bead or bead core. In some embodiments, the pharmaceutical dosage form herein can include a first immediate release bead or bead core wherein the CTN is present in an amount in a range of 5 wt. % to 15 wt. % based on the total weight of the immediate release bead or bead core and a second immediate release bead wherein the CTN is present in an amount in a range of 40 wt. % to 60 wt. % based on the total weight of the immediate release bead or bead core. In some embodiments, the pharmaceutical dosage form can include a bead core having CTN in an amount in a range of about 70 wt. % to about 90 wt. %, or about 75 wt. % to about 80 wt. % based on the total weight of the bead core, together with one or more coatings over the bead core.

In some embodiments, the sustained release beads can be present in the formulation or dosage form in an amount in a range of about 5% to about 80% based on the total weight of the plurality of CTN beads in the formulation or dosage form. For example, the sustained release beads are present in the dosage form in an amount in a range of about 5% to about 65%, or about 10% to about 60%, or about 20% to about 60%, or about 25% to about 55%, or about 25% to about 50%, or about 35% to about 55%, or about 40% to about 50%, or about 45% to about 55%, based on the total weight of the plurality of CTN beads. In some embodiments, the sustained release beads are present in the dosage form in an amount in a range of about 5% to about 65% based on the total weight of the plurality of CTN beads. In some embodiments, the sustained release beads are present in the dosage form in an amount in a range of about 35% to about 55% based on the total weight of the plurality of CTN beads, or about 42% to about 48%.

In some embodiments, the sustained release bead can include the CTN in an amount in a range of 10 wt. % to 95 wt. % in the sustained release bead based on the total weight of the sustained release bead. For example, the sustained release bead can include the CTN in an amount in a range of about 30 wt. % to about 90 wt. %, about 40 wt. % to about 90 wt. %, or about 50 wt. % to about 90 wt. %, or about 50 wt. % to about 80 wt. %, or about 50 wt. % to about 70 wt. %, based on the total weight of the sustained release beads. In some embodiments, the sustained release bead can include the CTN in an amount in a range of 40 wt. % to 90 wt. % in the sustained release bead based on the total weight of the sustained release bead. In embodiments, the sustained release bead can include the CTN in an amount in a range of 50 wt. % to 70 wt. % in the sustained release bead based on the total weight of the sustained release bead.

In some embodiments, the delayed release beads can be present in the formulation or dosage form in an amount in a range of about 5% to about 80% based on the total weight of the plurality of CTN beads in the formulation or dosage form. For example, the delayed release beads are present in the dosage form in an amount in a range of about 5% to about 65%, or about 10% to about 70%, or about 20% to about 60%, or about 25% to about 55%, or about 25% to about 50%, or about 30% to about 55%, or about 36% to about 46%, or about 40% to about 50%, based on the total weight of the plurality of CTN beads. In some embodiments, the delayed release beads are present in the dosage form in an amount in a range of about 5% to about 65% based on the total weight of the plurality of CTN beads. In some embodiments, the delayed release beads are present in the dosage form in an amount in a range of about 30% to about 55% based on the total weight of the plurality of CTN beads, or about 38% to about 44%.

In some embodiments, the delayed release bead can include the CTN in an amount in a range of 10 wt. % to 95 wt. % in the delayed release bead based on the total weight of the delayed release bead. For example, the delayed release bead can include the CTN in an amount in a range of about 30 wt. % to about 90 wt. %, about 40 wt. % to about 90 wt. %, or about 50 wt. % to about 90 wt. %, or about 50 wt. % to about 85 wt. %, or about 50 wt. % to about 70 wt. %, based on the total weight of the delayed release bead. In some embodiments, the delayed release bead can include the CTN in an amount in a range of 40 wt. % to 90 wt. % in the delayed release bead based on the total weight of the delayed release bead. In some embodiments, the delayed release bead can include the CTN in an amount in a range of 50 wt. % to 70 wt. % in the delayed release bead based on the total weight of the delayed release bead.

In some embodiments, the immediate release beads can be present in the formulation or dosage form in an amount in a range of about 1% to about 10% by weight of the formulation or dosage form, the sustained release beads can be present in the dosage form or dosage form in an amount of about 45% to about 55% by weight of the formulation or dosage form, and the delayed release beads can be present in the dosage form or dosage form in an amount in a range of about 40% to about 50% of the weight of the formulation or dosage form. In some embodiments, the immediate release beads can be present in the formulation or dosage form in an amount in a range of about 4% to about 28% by weight of the formulation or dosage form, the sustained release beads can be present in the formulation or dosage form in an amount of about 15% to about 40% by weight of the formulation or dosage form, and the delayed release beads can be present in the formulation or dosage form in an amount in a range of about 30% to about 65% of the weight of the formulation or dosage form.

In some embodiments, the immediate release beads can be present in the formulation or dosage form in an amount in a range of about 11% to about 17% by weight of the formulation or dosage form, the sustained release beads can be present in the formulation or dosage form in an amount of about 42% to about 48% by weight of the formulation or dosage form, and the delayed release beads (or delayed-sustained release beads) can be present in the formulation or dosage form in an amount in a range of about 38% to about 44% of the weight of the formulation or dosage form; such embodiments are particularly contemplated when the drug loading in each bead type is about 5 wt. % to about 10 wt. %, about 45 wt. % to about 55 wt. %, and about 45 wt. % to about 55 wt. %, based on the total weight of the beads, respectively. In embodiments, the immediate release beads can be present in the formulation or dosage form in an amount in a range of about 43% to about 49% by weight of the formulation or dosage form, the sustained release beads can be present in the formulation or dosage form in an amount of about 25% to about 31% by weight of the formulation or dosage form, and the delayed release beads can be present in the formulation or dosage form in an amount in a range of about 38% to about 44% of the weight of the formulation or dosage form; such embodiments are particularly contemplated when the drug loading in each bead type is about 45 wt. % to about 55 wt. %.

### Core Bead Formulation

The plurality of CTN beads each include a core particle. The core particle includes CTN and an excipient. The CTN bead can consist of an uncoated core particle itself. As further described below, CTN beads can include a core particle and one or more coatings.

In some embodiments, the core particles can be characterized by having a distribution of particle sizes. In embodiments, at least a portion of the core particles, or all of the core particles, of the plurality of CTN beads can have a core particle size (maximum diameter) of about 0.2 mm to about 2 mm, or about 0.3 mm to about 1.5 mm, about 0.4 mm to about 1.5 mm. For example, at least a portion of the core particles of the plurality of CTN beads have a core particle size of about 0.2 mm to about 2 mm, or about 0.3 mm to about 1.5 mm, about 0.4 mm to about 1.4 mm, or about 0.4 mm to about 1.3 mm, or about 0.4 mm to about 1.2 mm, or about 0.4 mm to about 1.1 mm, or about 0.5 mm to about 1 mm, or about 0.5 mm to 0.85 mm, or about 0.5 mm to about 0.71 mm. In embodiments at least a portion of the core particles of the plurality of CTN beads have a core particle size of about 0.5 mm to about 0.71 mm. In embodiments, the core particles of the plurality of CTN beads have a core particle size of about 0.5 mm to about 0.71 mm. Core particle sizes can be selected by sieving, for example, to reject particles having sizes outside the desired range. In embodiments, the distribution of particles sizes of the core particles can be characterized by at least 60% by weight of the core particles having a particle size in a range of about 0.4 mm to about 1.5 mm. For example, the distribution of particles sizes of the core particles can be characterized by at least 60% by weight of the core particles having a particle size in a range of about 0.4 mm to about 1.4 mm, or about 0.4 mm to about 1.3 mm, or about 0.4 mm to about 1.2 mm, or about 0.4 mm to about 1.1 mm, or about 0.5 mm to about 1 mm, or about 0.5 mm to 0.85 mm, or about 0.5 mm to about 0.71 mm. In embodiments, the distribution of particles sizes of the core particles is characterized by at least 60% by weight of the core particles having a particle size in a range of about 0.5 mm to about 0.71 mm. In some embodiments, the distribution of particles sizes of the core particles is characterized by at least 80% by weight of the core particles having a particle size in a range of about 0.4 mm to about 1.5 mm. For example, the distribution of particles sizes of the core particles is characterized by at least 80% by weight of the core particles having a particle size in a range of about 0.4 mm to about 1.4 mm, or about 0.4 mm to about 1.3 mm, or about 0.4 mm to about 1.2 mm, or about 0.4 mm to about 1.1 mm, or about 0.5 mm to about 1 mm, or about 0.5 mm to 0.85 mm, or about 0.5 mm to about 0.71 mm. In embodiments, the distribution of particles sizes of the core particles is characterized by at least 80% by weight of the core particles having a particle size in a range of about 0.5 mm to about 0.71 mm. In some embodiments, the distribution of particles sizes of the core particles is characterized by at least 90% by weight of the core particles having a particle size in a range of about 0.4 mm to about 1.5 mm. For example, the distribution of particles sizes of the core particles is characterized by at least 90% by weight of the core particles having a particle size in a range of about 0.4 mm to about 1.4 mm, or about 0.4 mm to about 1.3 mm, or about 0.4 mm to about 1.2 mm, or about 0.4 mm to about 1.1 mm, or about 0.5 mm to about 1 mm, or about 0.5 mm to 0.85 mm, or about 0.5 mm to about 0.71 mm. In embodiments, the distribution of particles sizes of the core particles is characterized by at least 90% by weight of the core particles having a particle size in a range of about 0.5 mm to about 0.71 mm. In some embodiments, the plurality of CTN beads can have a median particle size (diameter) in a range of about 0.2 mm to about 2.8 mm. For example, the plurality of CTN beads can have a median particle size (diameter) in a range of about 0.2 mm to about 2.5 mm, or about 0.2 mm to about 2.0 mm, or about 0.7 mm to about 2.5 mm, or about 0.7 mm to about 2.8 mm, or about 0.5 mm to about 2.8 mm, or about 0.8 mm to about 1.7 mm, or about 0.5 mm to about 1.2 mm, or about 0.5 mm to about 1.0 mm, or about 0.5 mm to about 0.71 mm. In some embodiments, the plurality of CTN beads can have a median particle size (diameter) in a range of about 0.5 mm to about 0.71 mm.

The amount of CTN in the core particles can be an amount in a range of about 5 wt. % to about 95 wt. %. In some embodiments, at least a portion of the plurality of CTN beads include core particles including the CTN in an amount in a range of about 5 wt. % to about 75 wt. %. For example, at least a portion of the plurality of CTN beads include core particles including the CTN in an amount in a range of about 5 wt. % to about 70 wt. %, or about 10 wt. % to about 70 wt. %, or about 20 wt. % to about 60 wt. %, or about 30 wt. % to about 60 wt. %, or about 40 wt. % to about 60 wt. %, or about 45 wt. % to about 55 wt. %. In some embodiments, at least a portion of the plurality of CTN beads include core particles including the CTN in an amount in a range of about 45 wt. % to about 55 wt. %. In some embodiments, at least a portion of the plurality of CTN beads include core particles including the CTN in an amount of about 50 wt. %. For example, immediate release beads can include a core particle including the CTN in an amount of about 10 wt. %, or about 50 wt. %. In some embodiments, at least a portion of the plurality of CTN beads include core particles including the CTN in an amount in a range of about 25 wt. % to about 95 wt. %. For example, at least a portion of the plurality of CTN beads include core particles including the CTN in an amount in a range of about 25 wt. % to about 90 wt. %, or about 35 wt. % to about 90 wt. %, or about 45 wt. % to about 90 wt. %, or about 50 wt. % to about 85 wt. %, or about 60 wt. % to about 85 wt. %, or about 75 wt. % to about 85 wt. %, or about 50 wt. %, about 60 wt. %, about 70 wt. %, or about 80 wt. %. In embodiments, at least a portion of the plurality of CTN beads include core particles including the CTN in an amount in a range of about 75 wt. % to about 85 wt. %. In some embodiments, at least a portion of the plurality of CTN beads include core particles including the CTN in an amount of about 80 wt. %. For example, sustained release beads can include a core particle including the CTN in an amount of about 80 wt. %. For example, the delayed release beads include a core particle including the CTN in an amount in a range of about 80 wt. %.

The core particles disclosed herein include an excipient. In some embodiments, the excipient includes one or more materials selected from a filler, a binder, a glidant, a surfactant, a polymer coating, a lubricant, a disintegrant, and a plasticizer. In some embodiments, the excipient can include one or more materials selected from a filler, a binder, a glidant, a surfactant, a polymer coating, and a plasticizer. In some embodiments, the excipient can include a filler and a binder. In some embodiments, the excipient can include a binder and a polymer coating. In some embodiments, the excipient can include a filler, a binder, and a polymer coating. In some embodiments, the excipient can include a filler, a binder, a polymer coating, and a plasticizer. In some embodiments, the pharmaceutical formulation can be free of disintegrants. In embodiments, a dosage form containing the pharmaceutical formulation can be free of disintegrants.

Fillers can include, but are not limited to, lactose, saccharose, glucose, starch, microcrystalline cellulose, microfine cellulose, mannitol, sorbitol, calcium hydrogen phosphate, aluminum silicate, amorphous silica, and sodium chloride, starch, and dibasic calcium phosphate dehydrate. In some embodiments, the filler is not water soluble, although it may absorb water. In some embodiments, the filler is a spheronization aid. Spheronization aids can include one or more of crospovidone, carrageenan, chitosan, pectinic acid, glycerides, β-CD, cellulose derivatives, microcrystalline cellulose, powdered cellulose, polyplasdone crospovidone, and polyethylene oxide, for example. In one type of embodiment, the filler includes microcrystalline cellulose.

Binders can include, but are not limited to, cellulose ethers, methyl cellulose, ethyl cellulose, hydroxyethyl cellulose, propyl cellulose, hydroxypropyl cellulose, lower-substituted hydroxypropyl cellulose, hydroxypropylmethyl cellulose (hypromellose, e.g. hypromellose 2910, METHOCEL™ E-5 [CAS 9004-65-3] same as hydroxypropylmethyl cellulose HPMC available from Sigma or DuPont™), carboxymethyl cellulose, starch, pregelatinized starch, acacia, tragacanth, gelatin, polyvinyl pyrrolidone (povidone, PVP), cross-linked polyvinyl pyrrolidone, sodium alginate, microcrystalline cellulose, and lower-substituted hydroxypropyl cellulose. In some embodiments, the binders are selected from wet binders. In some embodiments, the binder is selected from cellulose ethers, e.g. hypromellose.

Surfactants can include, but are not limited to, anionic surfactants, including sodium lauryl sulfate, sodium deoxycholate, dioctyl sodium sulfosuccinate, and sodium stearyl fumarate, nonionic surfactants, including polyoxyethylene ethers, and polysorbate 80, and cationic surfactants, including quaternary ammonium compounds. In some embodiments, the surfactant is selected from anionic surfactants, e.g. sodium lauryl sulfate.

Disintegrants can include, but are not limited to, starch, sodium cross-linked carboxymethyl cellulose, croscarmellose sodium, croscarmellose calcium, cross-linked polyvinyl pyrrolidone, and sodium starch glycolate, low-substituted hydroxypropyl cellulose, and hydroxypropyl starch.

Glidants can include, but are not limited to, polyethylene glycols of various molecular weights, magnesium stearate, calcium stearate, calcium silicate, fumed silicon dioxide, magnesium carbonate, magnesium lauryl sulfate, aluminum stearate, stearic acid, palmitic acid, cetanol, stearol, and talc.

Lubricants can include, but are not limited to, stearic acid, magnesium stearate, calcium stearate, aluminum stearate, and siliconized talc.

In some embodiments, the excipient can include one or more materials selected from lactose, mannitol, corn starch, microcrystalline cellulose, hydroxypropyl cellulose, hydroxypropyl methylcellulose (hypromellose), polyvinyl pyrrolidone, talc, polysorbate 80, glycerol monostearate, triethyl citrate, polyvinyl alcohol-polyethylene glycol graft copolymer (e.g., Kollicoat™ IR CAS 96734-39-3 available from Sigma) and silica. In some embodiments, the excipient can include microcrystalline cellulose and mannitol. In some embodiments, the excipient can include microcrystalline cellulose, talc, hypromellose, and polysorbate 80. In embodiments, the excipient can include microcrystalline cellulose. In some embodiments, the core particles can include an excipient containing microcrystalline cellulose.

The amount of filler in the core particle is not particularly limited. In some embodiments, the amount of filler (e.g. microcrystalline cellulose) can be in a range of about 10 wt. % to about 90 wt. %, about 10 wt. % to about 75 wt. %, or about 10 wt. % to about 60 wt. %, or at least 10 wt. %, or at least 15 wt. %, for example about 20 wt. %, or about 30 wt. %, or about 40 wt. %, or about 50 wt. %.

The amount of binder in the core particle is not particularly limited. In some embodiments, the amount of binder (e.g. hypromellose and/or polyvinyl alcohol-polyethylene glycol graft copolymer) can be in a range of about 1 wt. % to about 10 wt. %, or about 2 wt. % to about 8 wt. %, or about 4 wt. % to about 6 wt. %, for example about 5 wt. %.

The amount of surfactant, e.g. as a processing aid, in the core particle is not particularly limited. In some embodiments, the amount of surfactant (e.g. microcrystalline cellulose) can be in a range of about 0.1 wt. % to about 1 wt. %, or about 0.2 wt. % to about 0.8 wt. %, or about 0.4 wt. % to about 0.6 wt. %, for example about 0.5 wt. %.

### Coatings

In some embodiments, the disclosed extended-release dosage forms disclosed herein includes a plurality of beads wherein at least a portion of the plurality of beads is coated. In some embodiments, at least a portion of the plurality of beads can be uncoated. In embodiments, the coating can be one or more coatings selected from a delayed release coating, a sustained release coating, and a delayed-sustained release coating. In some embodiments, at least a portion of the plurality of beads can include a delayed release coating. In some embodiments, at least a portion of the plurality of beads can include a sustained release coating. In embodiments, at least a portion of the plurality of beads can include a delayed-sustained release coating.

The coating material disclosed herein, e.g. a polymer, can be a delayed release coating. In embodiments, the delayed release coating can be one that will dissolve in intestinal juices at a pH level higher than that of the stomach, e.g. a pH of 4.5 or greater, such as within the small intestine, and therefore permit release of the active substance in the regions of the small intestine, or later, and substantially not in the upper portion of the GI tract. In some embodiments, the enteric material begins to dissolve in an aqueous solution at pH between about 4.5 to about 5.5. In some embodiments, the delayed release material rapidly dissolves in an aqueous solution at pH of about 5. In another type of embodiment, the delayed release material rapidly dissolves in an aqueous solution at pH of about 5.5. For example, a pH-sensitive material can be selected such that it will not undergo significant dissolution until the dosage form has emptied from the stomach. The pH of the small intestine gradually increases from about 4.5 to about 6.5 in the duodenal bulb to about 7.2 in the distal portions of the small intestine (ileum). In some embodiments, the delayed release material will dissolve at a pH of at least 7, or at least 7.2, or at least 7.4, e.g. to target release in the distal portion of the small intestine or colon. In some embodiments, the delayed release material is insoluble in water and gastric juices, but will instead swell to provide a membrane through which the CTN active can diffuse. An insoluble polymer can also be selected such that it swells at a particular pH threshold, e.g. at a pH of at least 7, or at least 7.2, or at least 7.4, e.g. to target release in the distal portion of the small intestine or colon.

The delayed release coating materials can include, but are not limited to, one or more of the following: cross-linked polyvinyl pyrrolidone; non-cross linked polyvinylpyrrolidone; hydroxypropylmethyl cellulose phthalate, hydroxypropylmethyl cellulose acetate succinate, cellulose acetate succinate; cellulose acetate phthalate, hydroxypropylmethyl cellulose acetate succinate, cellulose acetate trimellitate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylatedivinylbenzene copolymer; polyvinylalcohols; polyoxyethyleneglycols; polyethylene glycol; sodium alginate; galactomannan; carboxypolymethylene; sodium carboxymethyl starch; copolymers of acrylic acid and/or methacrylic acid with a monomer selected from the following: methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate, or octadecyl acrylate, e.g. EUDRAGIT™-L and -S series, including L 100-55, L 30 D-55, L 100, S 100, L 12.5, and S 12.5, available from Evonik Industries of Essen, North Rhine-Westphalia, Germany; polyvinyl acetate; fats; oils; waxes; fatty alcohols; shellac; zein; gluten; ethylacrylate-maleic acid anhydride copolymer; maleic acid anhydride-vinyl methyl ether copolymer; styrol-maleic acid copolymer; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); and polyurethane.

A combination of delayed release coatings may also be used. In one type of embodiment, the delayed release coating dissolves at pH 7.0 or higher, or 7.2 or higher, or 7.4 or higher, e.g. to provide release in the colon. For example, the delayed release coating can be selected from a copolymer of methacrylic acid and methyl methacrylate, and a copolymer of methacrylic acid and ethyl acrylate. In some embodiments, the delayed release coating can include one or more materials selected from amylose acetate phthalate, cellulose acetate phthalate, cellulose acetate succinate, cellulose acetate trimellitate, carboxymethyl ethylcellulose, co-polymerized methacrylic acid/methacrylic acid methyl esters, co-polymerized methacrylic acid/methyl methacrylate, co-polymerized methylacrylate/methyl methacrylate/methacrylic acid, hydroxypropyl methylcellulose acetate succinate, hydroxypropyl methylcellulose phthalate, polyvinyl acetate phthalate, styrene maleic acid copolymer, styrene vinylpyridine copolymer. In embodiments, the delayed release coating can include one or more materials selected from a copolymer of methacrylic acid, methyl methacrylate, and methyl acrylate, and a methacrylic acid-acrylate copolymer. In embodiments, the delayed release coating can include a copolymer of methyl arylate, methyl methacrylate, and methacrylic acid, e.g. in a molar ratio of about 7:3:1 (e.g., Eudragit™ FS 30 D). Eudragit™ FS 30 D is poly(methyl acrylate-CO-methyl methacrylate-CO-methacrylic acid [CAS 26936-24-3] available from Evonik Industries. In other embodiments, the delayed release coating will not include cationic copolymers of ethyl acrylate, methyl methacrylate, and methacrylic acid ester with quaternary ammonium groups (e.g. Eudragit™ RL and Eudragit™ RS polymers). Eudragit™ RL 30 D and Eudragit™ RS 30 D are aqueous dispersions of copolymers of acrylic acid and methacrylic acid esters with a low content of quaternary ammonium groups available from Evonik Industries.

In some embodiments, the delayed release coating can also provide sustained release of CTN. In some embodiments, the delayed release coating will include an anionic polymer, optionally including carboxylate moieties. In some embodiments, the delayed release coating comprises a coating based on a copolymer of methyl acrylate, methyl methacrylate, and methacrylic acid, e.g. in a molar ratio of about 7:3:1 (e.g., Eudragit™ FS 30 D), and also has a sustained release function. Without intending to be bound by any particular theory, it is possible that since the poly(methyl acrylate-co-methyl methacrylate-co-methacrylic acid) polymer is anionic, having negatively charged carboxylate moieties (ratio of carboxyl groups to ester groups approx. 1:10), and centanafadine is a positively-charged secondary amine, the polymer could be influencing the rate of release of centanafadine from the beads due to ionic interaction.

Examples of some delayed release coatings are disclosed in U.S. Pat. No. 5,225,202, including beeswax and glyceryl monostearate; beeswax, shellac and cellulose; and cetyl alcohol, mastic and shellac, as well as shellac and stearic acid (U.S. Pat. No. 2,809,918); polyvinyl acetate and ethyl cellulose (U.S. Pat. No. 3,835,221); and neutral copolymer of polymethacrylic acid esters (Eudragit™ L30D) (F. W. Goodhart et al., Pharm. Tech., pp. 64-71, April 1984); copolymers of methacrylic acid and methacrylic acid methylester (Eudragit™ polymers), or a neutral copolymer of polymethacrylic acid esters containing metallic stearates (Mehta et al., U.S. Pat. Nos. 4,728,512 and 4,794,001). Such coatings comprise mixtures of fats and fatty acids, shellac and shellac derivatives and the cellulose acid phthalates, e.g., those having a free carboxyl content. See also Remington's Pharmaceutical Sciences, A. Osol, ed., Mack Pub. Co., Easton, Pa. (16th ed. 1980) at pages 1590-1593, and Zeitova et al. (U.S. Pat. No. 4,432,966), for descriptions of suitable enteric coating compositions.

The coating material disclosed herein, e.g. a polymer, can be a sustained release coating. A non-limiting list of suitable sustained release materials include hydrophilic and/or hydrophobic materials, such as gums, cellulose ethers, acrylic resins, protein derived materials, waxes, shellac, and oils such as hydrogenated castor oil and hydrogenated vegetable oil. However, any pharmaceutically acceptable hydrophobic or hydrophilic sustained release material which is capable of imparting sustained release of the CTN may be used in accordance with the present invention. In some embodiments, the sustained release coatings can include one or more materials selected from alkylcelluloses such as ethylcellulose, acrylic and methacrylic acid polymers and copolymers; and cellulose ethers, especially hydroxyalkylcelluloses (especially hydroxypropylmethylcellulose) and carboxyalkylcelluloses. Acrylic and methacrylic acid polymers and copolymers can include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. In some embodiments, the sustained release coating material is insoluble in water.

In some embodiments, the sustained release coating can include one or more materials selected from an alkylcellulose such as ethylcellulose, acrylic acid polymer, a methacrylic acid polymer, an acrylic acid copolymer, a methacrylic acid copolymer, and a cellulose ether hydroxyalkylcelluloses (especially hydroxypropyl methylcellulose) and carboxyalkylcelluloses. In embodiments, the sustained release coating can include one or more materials selected from a hydroxyalkylcellulose, a carboxyalkylcellulose, a methyl methacrylate, a methyl methacrylate copolymer, an ethoxyethyl methacrylate, an ethyl acrylate, a trimethyl ammonioethyl methacrylate, a cyanoethyl methacrylate, an aminoalkyl methacrylate copolymer, a poly(acrylic acid), a poly(methacrylic acid), a methacrylic acid alkylamine copolymer, a poly(methyl methacrylate), a poly(methacrylic acid)(anhydride), a polymethacrylate, polyacrylamide, a poly(methacrylic acid anhydride), and a glycidyl methacrylate copolymer. In some embodiments, the sustained release polymers can include one or more materials selected from poly[ethyl acrylate, methyl methacrylate, trimethylammonioethyl methacrylate chloride], hydroxypropylmethylcellulose, and poly[ethyl acrylate, methyl methacrylate]. In some embodiments, the sustained release polymer can include poly[ethyl acrylate, methyl methacrylate], e.g., in a molar ratio of about 2:1 (e.g., Eudragit™ NM 30 D). In embodiments, the sustained release coating can include one or more materials selected from the EUDRAGIT™-RL, -RS, -NE, and -NM series, including RL30D, RS30D, NE 30 D, NM 30 D, available from Evonik Industries. A combination of sustained release coatings may also be used. Eudragit™ NM 30D [CAS 9010-88-4] and Eudragit™ NE 30D [CAS 9010-88-2] are an aqueous dispersion of a neutral copolymer based on ethyl acrylate and methacrylate of approximately 30% polymer content available from Evonik Industries. In embodiments, the sustained release polymer can include ethylcellulose, e.g. Aquacoat™ ECD 30 D Coating available from DuPont™.

One or more plasticizers can be added to the delayed release coatings and/or sustained release coatings in order to increase their pliability and reduce brittleness, as it is known in the art. Suitable plasticizers are known in the art and include, for example, butyl citrates, triethyl citrate, diethyl phthalate, dibutyl sebacate, PEGs (e.g. PEG 6000), acetyl triethyl citrate, and triacetin. In some embodiments, the plasticizer is triethyl citrate. While some delayed release coatings and/or sustained release coatings are flexible and do not require addition of plasticizers, more brittle polymers (e.g., Eudragit™ L/S types, Eudragit™ RL/RS, and Eudragit™ FS 30 D) benefit from plasticizers, e.g. in the range of 5 wt. % to 30 wt. % based on the dry polymer mass, e.g. about 8 wt. % to about 12 wt. % of PlasACRYL™ T20 available from Evonik Industries, an anti-adherent system which contains glycerol monostearate, triethyl citrate and polysorbate 80 with a solid content of about 20%.

One or more anti-tacking agents (antiadherents) can also be added to an enteric coating mixture in order to reduce the tackiness of the film and prevent agglomeration, as it is known in the art. Anti-tacking agents include talc, and glyceryl monostearate, fumed silica (e.g., AEROSIL™ 200 available from Evonik Industries), precipitated silica (e.g., SIPERNAT™ PQ), and magnesium stearate, for example. Anti-tacking agents can be used in any suitable quantity, for example in a range of about 10 wt. % to 100 wt. % based on dry polymer mass, or about 1 wt. % to about 30 wt. %, or about 10 wt. % to about 50 wt. %, or about 10 wt. % to about 30 wt. %, or about 15 wt. % to about 30 wt. %. For example, in one embodiment the amount of talc is in a range of 15 wt. % to about 30 wt. %, based on dry polymer mass. In some embodiments, the amount of talc is in a range of 1 wt. % to about 10 wt. %, based on dry polymer mass.

One or more surfactants can also be added to the delayed release coating and/or the sustained release coating in order to improve substrate wettability and/or stabilize suspensions, as it is known in the art. Surfactants include polysorbate 80, sorbitan monooleate, and sodium dodecyl sulfate, for example.

The delayed release coating and/or the sustained release coating can be formed by any suitable process. Coating processes include pan coating, fluid bed coating, and dry coating (e.g., heat dry coating and electrostatic dry coating), for example. Pan coating and fluid bed coating using solvent are well established processes. In liquid coating, the enteric material and optional excipients (e.g. pigments, plasticizers, and/or anti-tacking agents) are mixed in an organic solvent or water to form a solution or dispersion. The coating solution or dispersion is sprayed into solid dosage forms in a pan coater or a fluid bed dryer and dried by hot air. For example, in a Wurster fluid bed coating process, the coating fluid is sprayed from the bottom of the fluid bed apparatus, whereas in an alternative the coating fluid is applied by top spraying, and in another alternative tangential spray is applied.

The amount of delayed release coating applied is sufficient to achieve desired release characteristics. For example, in one embodiment the amount of delayed release coating will be sufficient to meet United States Pharmacopeia (USP)<711> requirements (USP 43-NF 38 2S) for delayed-release dosage forms, by not releasing 10.0 wt. % of drug after 2 hours in 0.1N HCl. In another aspect, the formulation will be sufficient to release at least 80% of the active in the buffer stage, e.g. using the dissolution method of USP 43-NF 37 2S section <711>.

In some embodiments, the median amount of delayed release coating disposed over the core particle is at least 10 wt. % of the total weight of the CTN bead. In some embodiments, the median amount of delayed release coating disposed over the core particle is in a range of about 10 wt. % to about 50 wt. %, or about 10 wt. % to about 40 wt. %, or about 10 wt. % to about 30 wt. %, or about 20 wt. %, or about 12 wt. % to about 50 wt. %, or about 12 wt. % to about 35 wt. %, based on the total weight of the CTN bead. In some embodiments, the median amount of delayed release coating disposed over the core particle is in a range of about 15 wt. % to about 45 wt. %, based on the total weight of the CTN bead.

In some embodiments, the median amount of sustained release coating disposed over the core particle is at least 5 wt. % of the total weight of the coated CTN bead. In some embodiments, the median amount of sustained release coating disposed over the core particle is in a range of about 5 wt. % to about 50 wt. %, or about 7.5 wt. % to about 45 wt. %, or about 10 wt. % to about 40 wt. %, or about 15 wt. %, or about 5 wt. % to about 40 wt. %, 15 wt. % to about 40 wt. %, or about 20 wt. % to about 40 wt. %, based on the total weight of the coated CTN bead. In some embodiments, the median amount of sustained release coating disposed over the core particle is about 20 wt. % to about 40 wt. %, based on the total weight of the coated CTN bead.

In some embodiments, the median amount of sustained release coating disposed over the core particle is at least 5 wt. % by weight gain based total weight of the uncoated CTN bead. In some embodiments, the median amount of sustained release coating disposed over the core particle is in a range of about 5 wt. % to about 60 wt. %, or about 15 wt. % to about 60 wt. %, or about 20 wt. % to about 50 wt. %, or about 5 wt. % to about 40 wt. %, 15 wt. % to about 40 wt. %, or about 20 wt. % to about 40 wt. %, based on the total weight of the uncoated CTN bead. In some embodiments, the median amount of sustained release coating disposed over the core particle is about 20 wt. % to about 40 wt. %, based on the total weight of the uncoated CTN bead.

Additional lubricant (glidant, anti-tack agent) can be added to the coated beads in powder form. Anti-tacking agents include talc, glyceryl monostearate, fumed silica (e.g., AEROSIL™ 200), and precipitated silica (e.g., SIPERNAT™ PQ), for example. For example, talc powder can be added to the coated beads, optionally in an amount of 0.1 wt. % to about 3 wt. % based on the total bead weight.

Additionally, the coatings disclosed herein can further include a pore former. The rate of CTN release through the release coating materials can be low and be increased by the addition pore formers to the coating. Pore formers are often hydrophilic polymers, which dissolve in water and/or gastric media, to form pores in the coating layer. The amount and type of pore former material can be selected to affect the release profile and achieve a desired release profile. In embodiments, the pore former can include one or more materials selected from hydroxypropylcellulose, hydroxypropyl methylcellulose, polyethylene glycol, poloxamer 188, polyvinylpyrrolidone, d-mannitol, methyl cellulose, polyvinyl alcohol-polyethylene glycol graft copolymer, and a saccharide.

In embodiments, the pore former can include one or more materials selected from hydroxypropyl methylcellulose, hydroxypropylcellulose, and polyvinylpyrrolidone. In some embodiments, the pore former includes hydroxypropyl methylcellulose. In some embodiments, the pore former does not include polyvinylpyrrolidone. In some embodiments, the pore former does not include polyvinyl pyrrolidone when the release coating includes ethylcellulose. The pore former can be included in the release coating in an amount in a range of about 5 wt. % or more, or about 10 wt. % or more, or about 13 wt. % or more, or about 15 wt. % or more, or about 5 wt. % to about 20 wt. %, based on the total weight of the coating. In some embodiments, the pore former is present in an amount of about 15 wt. %, based on the total weight of the coating, or less than 50 wt. % or less than 20 wt. %% or in a range of about 1 wt. % to about 16 wt. %, or about 1 wt. % to about 12 wt. %, based on the total weight of the coating.

It is also contemplated herein that at least a portion of or the entirety of the plurality of beads includes a coating that includes only a soluble polymer which does not affect release of CTN from the formulation, such as a seal coating. In embodiments, the seal coating can include hydroxypropyl methylcellulose. In embodiments, the core beads can be coated with a seal coating prior to other coatings. In embodiments, at least a portion of the core particles are seal coated.

In some embodiments, the pharmaceutical formulation can include the plurality of CTN beads enclosed in one or more containers, for example selected from a capsule, sachet, and stick-pack. In some embodiments, the pharmaceutical formulation includes the plurality of CTN beads enclosed in a capsule. Soft and hard capsules are known. In some embodiments, the capsule is a hard capsule, e.g. a gelatin capsule or a vegetable-based hard capsule.

Thus, for example, some embodiments combining various of the features described above include a pharmaceutical formulation including a plurality of CTN beads, the beads including a core particle comprising CTN and a filler (optionally microcrystalline cellulose and/or mannitol), wherein the core particles are characterized by a distribution of particle sizes (maximum diameter) in a range of about 0.2 mm to about 1.5 mm, or about 0.3 mm to about 1.2 mm, or about 0.5 mm to about 0.85 mm, and wherein the core particles can include an optional coating surrounding the core particle, wherein the plurality of CTN beads includes an immediate release bead, a sustained release bead, and a delayed release bead.

A unit dosage form containing a CTN formulation according to the disclosure herein can include any suitable strength of CTN. For example, the amount of CTN in a unit dosage form can be in a range of about 1 mg to about 1800 mg, e.g., about 10 mg to about 1800 mg, e.g., about 25 mg to about 1800 mg, e.g., about 10 mg to about 1600 mg, e.g., about 10 mg to about 1200 mg, e.g., about 50 mg to about 490 mg, e.g., about 50 mg to about 250 mg, e.g., about 50 mg to about 1200 mg, e.g., about 50 mg to about 1000 mg, e.g., about 75 mg to about 1000 mg, e.g., about 75 mg to about 800 mg, e.g., about 75 mg to about 500 mg, e.g., about 100 mg to about 750 mg, e.g., about 100 mg to about 500 mg, e.g., about 100 mg to about 400 mg, e.g., about 100 mg to about 300 mg, e.g., about 100 mg to about 200 mg.

### Functional Characteristics

As mentioned above, the pharmaceutical formulation or dosage form can advantageously be designed to have one or more pharmacokinetic characteristics, e.g. in humans. The pharmaceutical formulations herein can be characterized by the amount of CTN released in vitro over a given time period. In embodiments wherein the pharmaceutical formulation includes immediate release beads, at least 90% of the CTN or salt thereof is released from the immediate release beads at a time in a range of 0 to 2 hours. In embodiments wherein the pharmaceutical formulation includes sustained release beads, at least 90% of the CTN is released from the sustained release beads at a time in a range of 2 to 6 hours. In embodiments wherein the pharmaceutical formulation includes delayed release beads, at least 90% of the CTN or salt thereof is released from the delayed release beads at a time in a range of 4 to 14 hours. In embodiments wherein the pharmaceutical formulation includes delayed-sustained release beads, at least 90% of the CTN or salt thereof is released from the delayed-sustained release beads at a time in a range of 4 to 14 hours.

In some embodiments, the formulation or dosage form can be characterized by one or more release profiles, in vivo and/or in vitro, selected from immediate release, sustained release, delayed release, and delayed-sustained release. In some embodiments, the formulation, e.g. one suitable for pediatric use, optionally can have a multiphasic release profile when tested in acid media for 2 hours followed by pH 7.4 buffered medium. For example, the release profile when determined according to USP<711> using Apparatus I (basket) in 1000 mL 0.1N hydrochloric acid at 37° C.+/−0.5° C. at 100 rpm for 2 hours, followed by Apparatus I (basket) in 1000 mL pH 7.4 phosphate buffer solution at 37° C.+/−0.5° C.) at 100 rpm for 16 hours can have a multiphasic release profile, optionally an at least biphasic release profile, optionally an at least triphasic release profile. Such a profile can optionally be characterized by release of about 22% to about 45% CTN at the 3-hour mark, further optionally by release of about 40% to about 65% of CTN at the 8-hour mark, and further optionally by release of about 65% to about 95% of CTN at the 12-hour mark, and further optionally by such rates of release at all three time points. In another type of embodiment, such a profile can be characterized by release of about 24% to 48% CTN at the 3-hour mark, further optionally by release of at least 66% CTN at the 6-hour mark, further optionally by release of at least 86% of CTN at the 10-hour mark, and further optionally by such rates of release at all three time points. Further optionally, the release profile can be characterized by a release of 49% to 73% at the 4-hour mark.

A pharmaceutical formulation herein optionally can be characterized by providing an in vivo absorption profile that is bimodal. In some embodiments, the pharmaceutical formulation with a bimodal in vivo absorption profile provides a first centanafadine plasma Cₘₐₓ at a time in a range of 0 to 4.5 hours, or about 0.5 hours to about 2 hours, or about 3.5 hours to about 4.5 hours. In some embodiments, the first centanafadine plasma Cₘₐₓ provided by the plurality of CTN beads is in a range of about 320 ng/mL to about 420 ng/mL, or about 325 ng/mL to about 390 ng/mL. In some embodiments, a pharmaceutical formulation with a bimodal in vivo absorption profile provides a second centanafadine plasma Cₘₐₓ at a time in a range of about 6 hours to 10 hours, or about 7 hours to 9 hours, or about 7.5 to about 8.5 hours. In some embodiments, the second centanafadine plasma Cₘₐₓ provided by the plurality of CTN beads is in a range of about 450 ng/mL to about 550 ng/mL, or about 470 ng/mL to about 530 ng/mL. In some embodiments, the in vivo absorption profile has a first centanafadine plasma Cₘₐₓ and a second centanafadine plasma Cₘax, wherein the first centanafadine plasma Cₘₐₓ and second centanafadine plasma Cₘₐₓ are separated by a time in a range of 1.5 to 8.5 hours, or about 2 hours to about 6 hours, or about 3 hours to about 5 hours.

A pharmaceutical formulation as described herein, and uses thereof, can be designed to provide one or more of the following pharmacokinetic profile characteristics.

The formulation can provide the subject with a relatively quick increase in plasma concentration of centanafadine to approach or meet a therapeutic concentration in a relatively short amount of time. Thus, for example, the formulation can provide an adult subject with a centanafadine plasma concentration at 1 hour post-dose (C₁ₕ) of at least 150 ng/mL, or at least 200 ng/mL, or at least 250 ng/mL, or at least 280 ng/mL, or in a range of about 180 ng/mL to about 610 ng/mL, or about 200 ng/mL to about 590 ng/mL, or about 220 ng/mL to about 540 ng/mL, or about 245 ng/mL to about 490 ng/mL; optionally such exposure can be achieved with a dosage strength in a range of about 145 mg to about 185 mg CTN, e.g. 164.4 mg CTN.

The formulation can provide an adult subject with a cumulative CTN plasma exposure in a subject at 1 hour post-dose (AUC₀₋₁ₕ) of at least 30 ng·h/mL, or at least 40 ng·h/mL, or at least 100 ng·h/mL, or at least 200 ng·h/mL, or in a range of about 30 ng·h/mL to about 500 ng·h/mL, or about 32 ng·h/mL to about 480 ng·h/mL, or about 36 ng·h/mL to about 440 ng·h/mL, or about 40 ng·h/mL to about 400 ng·h/mL; optionally such exposure can be achieved with a dosage strength in a range of about 145 mg to about 185 mg CTN, e.g. 164.4 mg CTN.

The formulation can maintain the plasma concentration of CTN in an adult subject in a therapeutic range for an extended period of time, for continuous efficacy. Thus, for example, the formulation can provide a CTN plasma concentration post-dose which remains at least at least 200 ng/mL, or at least 250 ng/mL, or at least 280 ng/mL, or at least 300 ng/mL, or at least 1000 ng/mL, or at least 1500 ng/mL, or in a range of about 150 ng/mL to about 4125 ng/mL, or about 160 ng/mL to about 3960 ng/mL, or about 180 ng/mL to about 3630 ng/mL, or about 200 ng/mL to about 3300 ng/mL over the time period 2 to 8 hours post-dose; optionally such plasma concentration can be achieved with a dosage strength in a range of about 145 mg to about 185 mg CTN, e.g. 164.4 mg CTN.

The formulation or use thereof can provide an adult subject with a cumulative CTN plasma exposure over the time period 0-8 hours post-dose (AUC₀₋₈ₕ) of at least 1275 ng h/mL, or at least 1530 ng·h/mL, or at least 1700 ng·h/mL, or at least 2500 ng·h/mL, or in a range of about 1275 ng·h/mL to about 6250 ng·h/mL, or about 1275 ng·h/mL to about 6250 ng·h/mL, or about 1360 ng·h/mL to about 6000 ng·h/mL, or about 1530 ng·h/mL to about 5500 ng·h/mL, or about 1700 ng·h/mL to about 5000 ng·h/mL; optionally such exposure can be achieved with a dosage strength in a range of about 145 mg to about 185 mg CTN, e.g. 164.4 mg CTN.

The formulation can provide a cumulative plasma exposure over the time period 2-8 hours post-dose (AUC₂₋₈ₕ) of at least 1050 ng·h/mL, or at least 1120 ng·h/mL, or at least 1330 ng·h/mL, or at least 2000 ng·h/mL, or at least 2500 ng·h/mL, or in a range of about 1050 ng·h/mL to about 5250 ng·h/mL, or about 1120 ng·h/mL to about 5040 ng·h/mL, or about 1260 ng·h/mL to about 4620 ng·h/mL, or about 1330 ng·h/mL to about 4410 ng·h/mL, or about 1400 ng·h/mL to about 4200 ng·h/mL; optionally such exposure can be achieved with a dosage strength in a range of about 145 mg to about 185 mg CTN, e.g. 164.4 mg CTN.

The pharmaceutical formulation or use thereof can provide an adult subject with a concentration of CTN in the plasma at 12 hours after administration (Cl₂ₕ) of at least 95 ng/mL, or at least 160 ng/mL, or at least 230 ng/mL, or at least 360 ng/mL, or in a range of about 95 ng/mL to about 450 ng/mL, or about 100 ng/mL to about 435 ng/mL, or about 110 ng/mL to about 400 ng/mL, or about 30 ng/mL to about 360 ng/mL; optionally such plasma concentration can be achieved with a dosage strength in a range of about 145 mg to about 185 mg CTN, e.g. 164.4 mg CTN.

The pharmaceutical formulation or use thereof can provide an adult subject with a relatively rapidly declining plasma concentration of CTN subsequent to 12 hours after administration, to promote a relatively low plasma concentration of CTN at 16 hours after administration and until the next dose. Thus, for example, the ratio of plasma concentration at 16 hours after administration to the plasma concentration at 12 hours after administration (C₁₆ₕ/C₁₂ₕ) can be less than 1, or 0.75 or less or 0.5 or less or 0.3 or less, or in a range of about 0.5 to 0.1; optionally such ratio can be achieved with a dosage strength in a range of about 145 mg to about 185 mg CTN, e.g. 164.4 mg CTN.

The pharmaceutical formulation or use thereof can provide an adult subject with a concentration of CTN in the plasma at 16 hours after administration of less than 375 ng/mL, or less than 300 ng/mL, or less 250 ng/mL, or less than 230 ng/mL, or less than 200 ng/mL, or less than 100 ng/mL, or in a range of about 60 ng/mL to about 375 ng/mL, or about 64 ng/mL to about 300 ng/mL, or about 76 ng/mL to about 250 ng/mL, or about 80 ng/mL to about 300 ng/mL; optionally such exposure can be achieved with a dosage strength in a range of about 145 mg to about 185 mg CTN, e.g. 164.4 mg CTN. For example, the plasma concentration can be relatively low at such a time to facilitate repeated once daily dosing without accumulation of CTN. In another aspect, the plasma concentration can be relatively low at such a time to avoid one or more adverse effects, e.g. insomnia in the subject, e.g. when the administration takes place in the morning.

The pharmaceutical formulation or use thereof can provide an adult subject with a cumulative CTN plasma exposure in the 24-hour period after administration (AUC₀₋₂₄ₕ) of at least 2400 ng·h/mL, or at least 2880 ng·h/mL, or at least 3200 ng·h/mL, or at least 5000 ng·h/mL, or at least 7100 ng·h/mL, or in a range of about 2400 ng·h/mL to about 12500 ng·h/mL, or about 2560 ng·h/mL to about 12000 ng·h/mL, or about 2880 ng·h/mL to about 11000 ng·h/mL, or about 3040 ng·h/mL to about 10500 ng·h/mL, or about 3200 ng·h/mL to about 10000 ng·h/mL, or about 7000 ng·h/mL to about 10000 ng h/mL. The pharmaceutical formulation or use thereof can provide an adult subject with a cumulative CTN plasma exposure in the 48-hour period after administration (AUC₀₋₄₈ₕ) of at least 2400 ng·h/mL, or 2880 ng·h/mL, or 3200 ng·h/mL, 5000 ng·h/mL, or 7100 ng h/mL, or in a range of about 2400 ng·h/mL to about 12500 ng·h/mL, or about 2560 ng·h/mL to about 12000 ng·h/mL, or about 2880 ng·h/mL to about 11000 ng·h/mL, or about 3040 ng·h/mL to about 10500 ng·h/mL, or about 3200 ng·h/mL to about 10000 ng h/mL, or about 7000 ng·h/mL to about 10000 ng·h/mL; optionally such exposure can be achieved with a dosage strength in a range of about 145 mg to about 185 mg CTN, e.g. 164.4 mg CTN.

The pharmaceutical formulation or use thereof can provide an adult subject with a cumulative CTN plasma exposure in the period after administration (AUC_{0-inf}) of at least 2400 ng·h/mL, or 2880 ng·h/mL, or 3200 ng·h/mL, 5000 ng·h/mL, or 7100 ng·h/mL, or in a range of about 2400 ng·h/mL to about 12500 ng·h/mL, or about 2560 ng·h/mL to about 12000 ng·h/mL, or about 2880 ng·h/mL to about 11000 ng·h/mL, or about 3040 ng·h/mL to about 10500 ng·h/mL, or about 3200 ng·h/mL to about 10000 ng·h/mL, or about 7000 ng·h/mL to about 10000 ng·h/mL; optionally such exposure can be achieved with a dosage strength in a range of about 145 mg to about 185 mg CTN, e.g. 164.4 mg CTN.

The pharmaceutical formulation or use thereof can provide an adult subject with a time until maximum CTN plasma concentration (tₘₐₓ) in a range of about 1.5 hours to about 11 hours, or about 2.25 hours to about 10 hours, or about 2.7 hours to about 8.8 hours, or about 3 hours to about 8 hours, or about 4 hours to about 6 hours.

The pharmaceutical formulations disclosed herein can be characterized by the mechanism of release of the active pharmaceutical ingredient (API), e.g., centanafadine hydrochloride. In some embodiments, one or more of the plurality of CTN beads has a release mechanism including one or more of dissolution, diffusion, erosion, osmosis, partitioning, swelling, and targeting. In some embodiments, one or more of the plurality of CTN beads has a diffusion release mechanism. In some embodiments, one or more of the plurality of CTN beads has a porous matrix leading to a diffusion release mechanism. In some embodiments, one or more of the plurality of CTN beads has a pH-triggered diffusion release mechanism. In some embodiments, one or more of the plurality of CTN beads has a combination of pH-triggered dissolution release mechanism and diffusion release mechanism. In some embodiments, as disclosed in part above, the delayed release beads have a combination of pH-triggered dissolution release mechanism and diffusion release mechanism. As used herein, the term “porous matrix” refers to an insoluble frame comprising a matrix of pores. In some embodiments, at least a portion of the plurality of beads include a porous matrix. In some embodiments, the sustained release beads comprise a porous matrix.

One type of pharmaceutical formulation disclosed herein comprises a plurality of centanafadine beads, the plurality of centanafadine beads each comprising a core particle comprising centanafadine or a pharmaceutically acceptable salt thereof and an excipient, wherein the plurality of centanafadine beads comprises a mixture of one or more immediate release beads, one or more sustained release beads, and one or more delayed release beads, at a ratio in a range of about 0.1-1:1-20:1-20 parts by weight, respectively.

The plurality of centanafadine beads can comprise the centanafadine or pharmaceutically acceptable salt thereof in an amount in a range of about 10 mg to about 490 mg.

The pharmaceutical formulation can comprise at least a portion of core particles comprising centanafadine or a pharmaceutically acceptable salt thereof in an amount in a range of about 70 wt. % to about 90 wt. %.

The pharmaceutical formulation can comprise core particles characterized by maximum diameter, of about 0.2 mm to about 2 mm.

The pharmaceutical formulation can comprise core particles wherein at least 60% by weight of the core particles, are characterized by maximum diameter, in a range of about 0.4 mm to about 1.5 mm.

The pharmaceutical formulation can comprise a plurality of centanafadine beads having a median particle size, characterized by the particle diameter, in a range of about 0.2 mm to about 2.8 mm.

The pharmaceutical formulation can comprise immediate release beads in a range of about 1% to about 75% based on the total weight of the plurality of centanafadine beads.

The pharmaceutical formulation can comprise sustained release beads in a range of about 5% to 80% based on the total weight of the plurality of centanafadine beads.

The pharmaceutical formulation can comprise delayed release beads in a range of about 5% to 80% based on the total weight of the plurality of centanafadine beads.

The pharmaceutical formulation can provide an adult subject with a centanafadine plasma concentration at 1 hour post-dose (C₁ₕᵣ) of at least 150 ng/mL, or in a range of about 150 ng/mL to about 610 ng/mL.

The pharmaceutical formulation can provide an adult subject with a centanafadine plasma concentration at 12 hours after administration (C₁₂ₕ) of at least 95 ng/mL, or in a range of about 95 ng/mL to about 450 ng/mL.

The pharmaceutical formulation can provide an adult subject with a centanafadine plasma concentration at 16 hours after administration (C₁₆ₕ) of less than 300 ng/mL, or in a range of about 95 ng/mL to about 300 ng/mL.

The pharmaceutical formulation can provide an adult subject with a centanafadine plasma concentration post-dose which remains at least 75 ng/mL, or in a range of about 75 ng/mL to about 1500 ng/mL over the time period 2 to 8 hours post-dose.

The pharmaceutical formulation can provide an adult subject with a cumulative centanafadine plasma exposure in the 24-hour period after administration (AUC₀₋₂₄ₕ) of at least 2400 ng·h/mL, or in a range of 2400 ng·h/mL to 12500 ng·h/mL.

The pharmaceutical formulation can provide an adult subject with a cumulative centanafadine plasma exposure after administration (AUC_{0-inf}) of at least 2400 ng·h/mL or in a range of 2400 ng·h/mL to 12500 ng·h/mL.

The pharmaceutical formulation can comprise centanafadine hydrochloride and provide an adult subject with a centanafadine plasma concentration at 1 hour post-dose (C₁ₕᵣ) of at least 150 ng/mL, a centanafadine plasma concentration post-dose which remains at least 250 ng/mL over the time period 2 to 8 hours post-dose, and a concentration of centanafadine in the plasma at 16 hours after administration (C₁₆ₕ) of less than 300 ng/mL.

The pharmaceutical formulation can comprise at least 40% of the centanafadine or salt thereof released from the mixture of beads at a time in a range of 3 hours to 5 hours, and at least 90% of the centanafadine or salt thereof released from the mixture of beads at a time in a range of 12 hours to 14 hours, according to the United States Pharmacopeia <711> with Apparatus 1 (basket) at 37° C.+/−0.5° C. at 100 rpm, first in 1000 ml of a 0.1N HCl solution for 2 hours, then 1000 ml pH 7.4 buffered water at 37° C.+/−0.5° C. at 100 rpm for the remainder of the time.

The pharmaceutical formulation can provide an adult subject with a centanafadine plasma concentration at 1 hour post-dose (C₁ₕᵣ) in a range of about 150 ng/mL to about 610 ng/mL; at 12 hours post-dose (C₁₂ₕ) in a range of about 95 ng/mL to about 450 ng/mL; at 16 hours post-dose (C₁₆ₕ) in a range of about 95 ng/mL to about 300 ng/mL; and the ratio wherein the ratio C₁₆ₕ/C₂ₕ is less than 1.

The pharmaceutical formulation can comprise at least a portion of sustained release beads comprising a coating which is a sustained release coating, and at least a portion of the delayed release beads comprising a delayed release coating and/or a delayed-sustained release coating.

The pharmaceutical formulation can comprise a delayed release coating comprising one or more materials selected from amylose acetate phthalate, cellulose acetate phthalate, cellulose acetate succinate, cellulose acetate trimellitate, carboxymethyl ethylcellulose, co-polymerized methacrylic acid/methacrylic acid methyl esters, copolymerized methacrylic acid/methyl methacrylate, co-polymerized methylacrylate/methyl methacrylate/methacrylic acid, hydroxypropyl methylcellulose acetate succinate, hydroxypropyl methylcellulose phthalate, polyvinyl acetate phthalate, styrene maleic acid copolymer, and styrene vinylpyridine copolymer.

The pharmaceutical formulation can comprise a sustained release coating comprising one or more materials selected from an alkylcellulose, acrylic acid polymer, a methacrylic acid polymer, an acrylic acid copolymer, a methacrylic acid copolymer, and a cellulose ether. The sustained-release coating further comprises a pore former. The pore former comprises one or more materials selected from hydroxypropylcellulose, hydroxypropyl methylcellulose, polyethylene glycol, poloxamer 188, polyvinylpyrrolidone, d-mannitol, methyl cellulose, polyvinyl alcohol-polyethylene glycol graft copolymer, and saccharide.

The pharmaceutical formulation can comprise a plurality of centanafadine beads, the plurality of centanafadine beads each comprising a core particle comprising centanafadine or a pharmaceutically acceptable salt thereof and an excipient, wherein the plurality of centanafadine beads comprises a mixture of one or more immediate release beads, one or more sustained release beads, and one or more delayed release beads, at ratio in a range of about 0.1-1:1-20:1-20 parts by weight, wherein the plurality of centanafadine beads comprises the centanafadine or pharmaceutically acceptable salt thereof in an amount in a range of about 10 mg to about 490 mg, wherein at least a portion of the sustained release beads comprises a coating which is a sustained release coating comprising an ethyl acrylate and methyl methacrylate copolymer; wherein at least a portion of the delayed release beads comprises a delayed release coating comprising poly(methyl acrylate-co-methyl methacrylate-co-methacrylic acid; and wherein the plurality of centanafadine beads has a median particle size, characterized by the particle diameter, in a range of about 0.2 mm to about 2.8 mm.

### Major Depressive Disorder (MDD)

The cause of depression is due to a deficiency or imbalance in the monoamine neurotransmitters, such as serotonin, dopamine and norepinephrine.

Depression diagnosis requires five or more symptoms (Diagnostic and Statistical Manual of Mental Disorders-DSM-5). Frontiers in Psychiatry, 2018. Vol. 9, Art. 450. One of them must be either Depressed mood or Anhedonia, named main criteria. Although the secondary symptoms can be divided into somatic and non-somatic clusters, the DSM-5 identify depression in all or none fashion. In contrast, depression severity is a continuous variable. The diagnosis of a Major Depression Episode (MDE) requires five or more symptoms to be present within a 2-week period. One of the symptoms should, at least, be either a depressed mood (DM) or anhedonia (loss of interest or pleasure-LI). The secondary symptoms of MDE are appetite or weight changes (AW), sleep difficulties (insomnia or hypersomnia), psychomotor agitation or retardation (PAR), fatigue or loss of energy (FE), diminished ability to think or concentrate (C), feelings of worthlessness or excessive guilt (FW), and suicidality (SU). These symptoms are rated in an all or none (0 or 1) fashion.

According to the DSM-5 criteria, the symptoms are summed to determine the presence or the absence of a major depression episode.

The disclosure provides methods for treating MDD and/or alleviating, improving or relieving MDD symptoms in a human patient in need thereof, comprising administering to said patient a therapeutically effective amount of (1R,5S)-1-(naphthalen-2-yl)-3-azabicyclo[3.1.0]hexane or a pharmaceutically acceptable salt thereof, wherein CTN is administered in an extended-release dosage form comprising a plurality of CTN beads, the plurality of CTN beads each comprising a core particle comprising CTN or a pharmaceutically acceptable salt thereof, and an excipient, and wherein the plurality of CTN beads comprises a mixture of one or more immediate release (IR) beads, one or more sustained release (SR) beads, and one or more delayed release (DR) beads.

The disclosure provides methods for treating MDD and/or alleviating, improving or relieving MDD symptoms in a human in need thereof, comprising administering to said patient a therapeutically effective amount of (1R,5S)-1-(naphthalen-2-yl)-3-azabicyclo[3.1.0]hexane or a pharmaceutically acceptable salt thereof, as adjunct to escitalopram, wherein CTN is administered in an extended-release dosage form.

### Dosage

As described herein, the methods disclosed herein comprise administering a therapeutically effective amount of CTN or a pharmaceutically acceptable salt thereof in an amount of about 10 mg or more, for example, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or about 400 mg or more of CTN or a pharmaceutically acceptable salt thereof per day. Further, the disclosed methods and uses can comprise administering CTN or a pharmaceutically acceptable salt thereof in an amount of about 750 mg or less per day, for example, 750, 740, 730, 720, 710, 700, 690, 680, 670, 660, 650, 640, 630, 620, 610, 600, 590, 580, 570, 560, 550, 540, 530, 520, 510, 500, 490, 480, 470, 460, 450, 440, 430, 420, or about 410 mg or less per day of CTN or a pharmaceutically acceptable salt thereof per day. Thus, the methods and uses disclosed herein can comprise administering CTN or a pharmaceutically acceptable salt thereof in an amount bounded by any of the aforementioned values, for example, in a range of about 10 mg to about 750 mg per day (e.g., about 10-750 mg, about 20-740 mg, about 30-730 mg, about 40-720 mg, about 50-710 mg, about 60-700, about 70-690 mg, about 80-680 mg, about 90-670 mg, about 100-660 mg, about 110-650 mg, about 120-640 mg, about 130-630 mg, about 140-620 mg, about 150-610 mg, about 160-600 mg, about 170-590 mg, about 180-580 mg, about 190-570 mg, about 200-560 mg, about 210-550 mg, about 220-540 mg, about 230-530 mg, about 240-520 mg, about 250-510 mg, about 260-500 mg, about 270-490 mg, about 280-480 mg, about 290-470 mg, about 300-460 mg, about 310-450 mg, about 320-440 mg, about 330-430 mg, about 340-420 mg, about 350-410 mg, about 360-400 mg, about 370-410 mg, or from about 380 to about 400 mg CTN or a pharmaceutically acceptable salt thereof per day). In embodiments, optionally in conjunction with other above or below embodiments, the amount of CTN or a pharmaceutically acceptable salt thereof administered is in a range of from about 10 to about 750 mg daily, for example, from about 50 to about 600 mg daily, from about 60 to about 450 mg daily, or from about 200 to about 400 mg daily.

The disclosed methods for treating MDD contemplate oral administration at dosages as described above, and in addition or in the alternative based on weight, e.g. in an amount in a range of about 0.01 to about 2.0 mg/kg. An indicated daily dosage for oral administration can be in the range of from about 0.10 mg to about 200 mg, conveniently administered once daily, or in divided doses 2 to 4 times daily, or in extended-release form. For example, a unit dosage form for oral administration can comprise from about 20 mg to about 75 mg or about 150 mg, e.g., from about 10 mg or about 20 mg or about 50 mg or about 75 mg or about 100 mg to 200 mg or about 500 mg of CTN, in free or pharmaceutically acceptable salt form, together with a pharmaceutically acceptable diluent or carrier therefor.

In embodiments, the disclosed methods can comprise administering about 200 mg CTN daily. In embodiments, the disclosed methods can comprise administering about 400 mg CTN daily. In embodiments, the disclosed methods can comprise administering about 41.1 mg CTN daily. In embodiments, the disclosed methods can comprise administering about 82.2 mg CTN daily. In embodiments, the disclosed methods can comprise administering about 123.3 mg CTN daily. In embodiments, the disclosed methods can comprise administering about 164.4 mg CTN daily. In embodiments, the disclosed methods can comprise administering about 246.6 mg CTN daily. In embodiments, the disclosed methods can comprise administering about 328.8 mg CTN daily. A dose according to any of the foregoing daily doses can be administered in a single unit dose per day.

These and other effective dosage amounts may be administered in a single dose, or in the form of multiple daily, weekly or monthly doses, for example in a dosing regimen comprising from 1 to 5, or 2-3, doses administered per day, per week, or per month to provide the desired TDD of CTN, as described herein.

In embodiments, optionally in conjunction with other above or below embodiments, the CTN or a pharmaceutically acceptable salt thereof can be administered one to four times a day.

In embodiments, optionally in conjunction with other above or below embodiments, the CTN or a pharmaceutically acceptable salt thereof is administered twice per day.

In embodiments, optionally in conjunction with other above or below embodiments, the CTN or a pharmaceutically acceptable salt thereof is administered once per day.

As described herein, in embodiments, CTN dosages can be calculated based on body weight, and can be administered, for example, in amounts from about 0.5 mg/kg to about 30 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

As described herein, the methods disclosed comprise administering a therapeutically effective amount of CTN or a pharmaceutically acceptable salt thereof as adjunct to escitalopram or to a pharmaceutically acceptable salt thereof.

As described herein, the methods disclosed comprise administering a therapeutically effective amount of CTN or a pharmaceutically acceptable salt thereof in a range of from about 10 to about 750 mg daily, for example, from about 35 to about 400 mg daily, from about 50 to about 600 mg daily, from about 60 to about 450 mg daily, or from about 200 to about 400 mg daily, as adjunct to escitalopram or to a pharmaceutically acceptable salt thereof in amount of about 2.5 to about 20 mg daily, for example, from about 5 to about 20 mg daily, from about 5 to about 10 mg daily, or from about 10 to about 20 mg daily. The methods disclosed comprise administering a therapeutically effective amount of CTN or a pharmaceutically acceptable salt thereof in a range of from about 10 to about 750 mg daily, as adjunct to escitalopram or to a pharmaceutically acceptable salt thereof in amount of about 2.5 to about 20 mg daily. The methods disclosed comprise administering a therapeutically effective amount of CTN or a pharmaceutically acceptable salt thereof in a range of from about 35 to about 400 mg daily, as adjunct to escitalopram or to a pharmaceutically acceptable salt thereof in amount of about 10 to about 20 mg daily. These dosages can be in the form of a single oral dosage form or separate dosage forms administered simultaneously or sequentially.

As described herein, the methods disclosed comprise administering a therapeutically effective amount of CTN as hydrochloride salt. As described herein, the methods disclosed comprise administering a therapeutically effective amount of escitalopram as hydrobromide salt. As described herein, the methods disclosed comprise administering a therapeutically effective amount of escitalopram as oxalate salt.

As described herein, the methods disclosed comprise administering orally a therapeutically effective amount of CTN hydrochloride salt as adjunct to a therapeutically effective amount of escitalopram oxalate or escitalopram hydrobromide.

The therapeutically effective amount of CTN hydrochloride salt is in a range from about from 35 to about 400 mg daily, and the therapeutically effective amount of escitalopram as oxalate or as hydrobromide salt is in a range from 2.5 to 20 mgs daily.

The amount, timing and mode of delivery of compositions of the invention comprising an effective amount of a compound of the present invention can be routinely adjusted on an individual basis, depending on such factors as weight, age, gender, and condition of the individual, the acuteness of the targeted condition, disorder and/or related symptoms, whether the administration is prophylactic or therapeutic, and on the basis of other factors known to effect drug delivery, absorption, pharmacokinetics, including half-life, and efficacy. An effective dose or multi-dose treatment regimen using the compounds described herein will ordinarily be selected to approximate a minimal dosing regimen that is necessary and sufficient to substantially treat, prevent, aid, or alleviate one or more symptom(s) of major depressive disorder.

Thus, for example, following administration of CTN or a pharmaceutically acceptable salt thereof in a method for treating MDD or for alleviating, improving or relieving MDD symptoms, a patient can exhibit a 10%, 20%, 30%, 50% or greater reduction, up to a 75-90%, or 95% or greater, reduction, in one or more symptoms associated with MDD, compared to placebo-treated or other suitable control subjects.

Following administration of CTN or a pharmaceutically acceptable salt thereof as adjunct to escitalopram or a pharmaceutically acceptable salt thereof, in a method for treating MDD or for alleviating, improving or relieving MDD symptoms, a patient can exhibit a 10%, 20%, 30%, 50% or greater reduction, up to a 75-90%, or 95% or greater, reduction, in one or more symptoms associated with MDD, compared to placebo-treated or other suitable control subjects.

### Route of Administration

The disclosed methods and uses comprise administering CTN or a pharmaceutically acceptable salt thereof in a suitable form for the desired route of administration. By way of example, CTN, in free or pharmaceutically acceptable salt form, can be administered by any suitable route, including orally, buccally, nasally, via aerosol, topical, mucosal, injectable, parenterally, transdermally, or by inhalation, including by sustained release, although various other known delivery routes, devices, and methods can likewise be employed.

In embodiments, optionally in conjunction with other above or below embodiments, the CTN or a pharmaceutically acceptable salt thereof (e.g., CTN) is administered intravenously, or transdermally (e.g., a transdermal patch and optionally one comprising microneedles), or orally.

In embodiments, optionally in conjunction with other above or below embodiments, the CTN or a pharmaceutically acceptable salt thereof is administered orally. Suitable nonlimiting examples of oral dosage forms include capsules or tablets. In some embodiments, the dosage form is a capsule.

In embodiments, CTN in free or pharmaceutically acceptable salt form, can be released from a composition and delivered into the blood plasma or other target site of activity in the subject (including, but not limited to, areas of the brain such as the prefrontal cortex, frontal cortex, thalamus, striatum, ventral tegmental area, other cortical areas, hippocampus, hypothalamus, or nucleus accumbens) in a sustained release profile. One type of sustained release profile can be characterized by providing in vitro release (according to USP<711> using Apparatus I (basket) in 1000 mL 0.1N hydrochloric acid at 37° C.+/−0.5° C. at 100 rpm for 2 hours, followed by Apparatus I (basket) in 1000 mL pH 7.4 phosphate buffer solution at 37° C.+/−0.5° C.) at 100 rpm for 16 hours). Another type of sustained release profile can be characterized by providing in vitro release (according to USP<711> using Apparatus I (basket) in 1000 mL 0.1N hydrochloric acid at 37° C.+/−0.5° C. at 100 rpm for 2 hours, followed by Apparatus I (basket) in 1000 mL pH 7.4 phosphate buffer solution at 37° C.+/−0.5° C.) at 100 rpm for 16 hours) which is multiphasic, optionally an at least biphasic. Such a profile can optionally be characterized by release of about 22% to about 45% CTN at the 3 hour mark, further optionally by release of about 40% to about 65% of CTN at the 8 hour mark, and further optionally by release of about 65% to about 95% of CTN at the 12 hour mark, and further optionally by such rates of release at all three time points. Alternatively, such a profile can be characterized by release of about 24% to 48% CTN at the 3 hour mark, further optionally by release of at least 66% CTN at the 6 hour mark, further optionally by release of at least 86% of CTN at the 10 hour mark, and further optionally by such rates of release at all three time points. Further optionally, the release profile can be characterized by a release of 49% to 73% at the 4 hour mark. In another aspect, the release profile can be determined in vivo, and characterized in that about 0% to 20% of the active compound is released and delivered (as determined, e.g., by measuring blood plasma levels) within 0 to 2 hours, from 20% to 50% of the active compound is released and delivered within about 2 to 12 hours, from 50% to 85% of the active compound is released and delivered within about 3 to 20 hours, and greater than 75% of the active compound is released and delivered within about 5 to 18 hours.

In embodiments, the in vivo absorption profile of the pharmaceutical formulation can have a concentration of CTN in the plasma at 16 hours after administration of less than 300 ng/mL, or less 250 ng/mL, or less than 230 ng/mL, or less than 200 ng/mL. For example, the plasma concentration can be relatively low at such a time to facilitate repeated once daily dosing without accumulation of CTN. In another aspect, the plasma concentration can be relatively low at such a time to avoid insomnia in the subject, e.g. when the administration takes place in the morning.

In embodiments, the Cₘₐₓ of CTN in free or pharmaceutically acceptable salt form, provided after administration of a sustained release pharmaceutical composition comprising CTN in free or pharmaceutically acceptable salt form, can be less than about 80%, e.g., less than about 75%, e.g., less than about 60%, e.g., less than about 50%, e.g., less than about 40%, e.g., less than about 30% of the Cₘₐₓ obtained after administering an equivalent dose of CTN in free or pharmaceutically acceptable salt form, in an immediate release pharmaceutical composition. In embodiments, the Cₘₐₓ of CTN in free or pharmaceutically acceptable salt form, provided after administration of a sustained release pharmaceutical composition comprising CTN in free or pharmaceutically acceptable salt form, can be about 20-80%, optionally about 30-80%, optionally about 20-70% optionally about 30-70%, optionally about 30-60%, optionally about 30-50%, optionally about 30-40%, of the Cₘₐₓ obtained after administering an equivalent dose of CTN in free or pharmaceutically acceptable salt form, in an immediate release pharmaceutical composition. In embodiments, the Cₘₐₓ CTN in free or pharmaceutically acceptable salt form, provided after administration of a sustained release pharmaceutical composition comprising CTN in free or pharmaceutically acceptable salt form, can be less than about 50%, optionally less than about 40%, optionally less than about 30%, of the Cₘₐₓ obtained after administering an equivalent dose of CTN in free or pharmaceutically acceptable salt form, in an immediate release pharmaceutical composition. In embodiments, the Cₘₐₓ CTN in free or pharmaceutically acceptable salt form, provided after administration of a sustained release pharmaceutical composition comprising CTN in free or pharmaceutically acceptable salt form, can be about 20-50%, optionally is about 30-50%, optionally is about 30-40%, of the Cₘₐₓ obtained after administering an equivalent dose of CTN in free or pharmaceutically acceptable salt form, in an immediate release pharmaceutical composition.

The composition of the immediate release centanafadine core beads, 50 nominal wt. % CTN HCl used in these formulations is outlined in Table 1.

TABLE 1
Composition of CTN HCl Core Beads
Composition of CTN HCl Core Beads

| Component | Wt. % |
|---|---|
| Centanafadine Hydrochloride | 49.75 |
| Microcrystalline Cellulose USP/NF, Ph. Eur, JP (Avicel ™ | 39.8 |
| PH-101 available from Dupont ™) | |
| Mannitol USP/NF, Ph. Eur, JP | 9.95 |
| (Pearlitol ™ 50 C available from Roquette) | |
| Talc, 194 Pharma M | 0.50 |
| Purified Water * | N/A |

* Purified water is dispensed in the manufacturing process depending on the needs of the batch size and is removed during the manufacturing process.

The compositions of the immediate release centanafadine HCl core beads, 10 wt % and 50 wt. % in these formulations are outlined in Table 2.

TABLE 2
Composition of immediate release beads

| | | Quantity (wt. % based on | |
|---|---|---|---|
| | Compendial | total weight of beads) | |
| Component | Standard | 10% DL | 50% DL |
| Centanafadine | In-house | 10.0 | 50.0 |
| Hydrochloride | | | |
| Microcrystalline Cellulose | USP/NF, EP, | 40.0 | — |
| (Avicel ® PH-301 available | JP | | |
| from Dupont ™) | | | |
| Microcrystalline Cellulose | USP/NF, EP, | — | 40.0 |
| (Avicel ™ PH-101) | JP | | |
| Mannitol (Pearlitic ™ 50C) | UP/NF, EP, JP | 50.0 | 10.0 |
| Purified Water* | USP | N/A | N/A |

*Purified water is dispensed in the manufacturing process depending on the needs of the batch size and is removed during the manufacturing process.

The composition of Centanafadine Core Beads (80 wt. % drug load) is provided in Table 3. Centanafadine Core Beads were manufactured by weighing out and dry blending the CTN HCl and microcrystalline cellulose in a high shear granulator and granulating the blended mixture with purified water, extruding and spheronization to form wet beads, fluid bed drying of the core beads, and sieving the beads to retain a desired size range.

TABLE 3
Composition of Centanafadine Core Beads, 80 wt. %

| | Compendial | Quantity (wt. % based on |
|---|---|---|
| Component | Standard | total weight of beads) |
| Centanafadine | In-house | 80.0 |
| Hydrochloride | | |
| Microcrystalline Cellulose | USP/NF, EP, JP | 20.0 |
| (Avicel ™ PH-301) | | |
| Purified Water* | USP | N/A |

*Purified water is dispensed in the manufacturing process depending on the needs of the batch size and is removed during the manufacturing process
N/A: not applicable

The composition of Centanafadine Seal Coated Beads (80 wt. % drug load) is provided in Table 4. For seal coating, the desired quantity of the hypromellose solution in water was sprayed using a Wurster process at a controlled set of process parameters, and then the coated beads were dried to a desired moisture content and cured.

TABLE 4
Composition of Centanafadine Seal Coated Beads, 80 wt. % active

| | Compendial | Quantity |
|---|---|---|
| Component | Standard | % w/w |
| Centanafadine Core Beads, | In-house | 97.1 |
| 80 wt. % active | | |
| Hypromellose | USP/NF, EP | 2.9 |
| (Methocel ™E-5) | | |
| Purified Water* | USP | N/A |

*Purified water is dispensed in the manufacturing process depending on the needs of the batch size and is removed during the manufacturing process.

The compositions of sustained release coated beads are provided in Table 5.

TABLE 5
Compositions of sustained release coated beads

| | | Quantity % w/w | | |
|---|---|---|---|---|
| | Compendial | 10% | 15% | 40% |
| Component | Standard | WG | WG | WG |
| Centanafadine Seal | In-house | 81.6 | 74.8 | 52.6 |
| Coated Beads, 80 wt. % | | | | |
| active | | | | |
| Ethyl Acrylate and Methyl | NF, EP | 8.2 | 11.2 | 21.1 |
| Methacrylate Copolymer | | | | |
| Disp. (Eudragit ™ NM 30D) | | | | |
| Hypromellose (Methocel ™ | USP/NF, EP | 1.2 | 1.7 | 3.2 |
| E-5) | | | | |
| Talc, 194 Pharma M | USP, EP, JP | 8.2 | 11.2 | 21.1 |
| Polysorbate 80 (Tween | NF, EP, JP | 0.8 | 1.1 | 2.1 |
| 80HP-LQ-MH) | | | | |
| Purified Water* | USP | N/A | N/A | N/A |

*Purified water is dispensed in the manufacturing process depending on the needs of the batch size and is removed during the manufacturing process.

The compositions of delayed release coated beads are provided in Table 6.

TABLE 6
compositions of delayed release coated beads

| | | Quantity % w/w | | | | |
|---|---|---|---|---|---|---|
| | Compendial | 10% | 15% | 20% | 30% | 40% |
| Component | Standard | WG | WG | WG | WG | WG |
| Centanafadine Seal | In-house | 90.1 | 85.8 | 82.0 | 75.2 | 69.4 |
| Coated Beads, 80 wt. % | | | | | | |
| active | | | | | | |
| Poly(Methyl Acrylate- | In-house | 9.0 | 12.9 | 16.4 | 22.6 | 27.8 |
| CO-Methyl | | | | | | |
| Methacrylate-CO- | | | | | | |
| Methacrylic Acid) | | | | | | |
| (Eudragit ™ FS 30 D, | | | | | | |
| Aq. Dispersion) | | | | | | |
| PlasACRYL ™ T20 | In-house | 0.9 | 1.3 | 1.6 | 2.3 | 2.8 |
| (emulsion of glyceryl | | | | | | |
| monostearate, triethyl | | | | | | |
| citrate, polysorbate 80 | | | | | | |
| and water with a solid | | | | | | |
| content of about 20%) | | | | | | |
| Purified Water* | USP | | | N/A | | N/A |

*Purified water is dispensed in the manufacturing process depending on the needs of the batch size and is removed during the manufacturing process.

The compositions of various centanafadine extended-release capsules are provided in Table 7, Table 8, and Table 9.

TABLE 7
Examples of centanafadine extended-release capsules

| | 52.5 mg | 78.8 mg | 210 mg | 241.5 mg |
|---|---|---|---|---|
| | Sustained | Sustained | Sustained | Sustained |
| | Release | Release | Release | Release |
| | Capsules | Capsules | Capsules | Capsules |
| | Quantity/ | Quantity/ | Quantity/ | Quantity/ |
| | Capsule | Capsule | Capsule | Capsule |
| Component | (mg) | (mg) | (mg) | (mg) |
| Centanafadine | 52.5 | 78.8 | 210 | 241.5 |
| hydrochloride | | | | |
| Mannitol | 12.5 | 19 | 2 | 2.3 |
| (PEARLITOL ™ 50C) | | | | |
| Microcrystalline | 22.6 | 33.9 | 50 | 57.5 |
| Cellulose (Avicel ™ PH- | | | | |
| 301) | | | | |
| Microcrystalline | — | — | 8 | 9.2 |
| Cellulose (Avicel ™ PH- | | | | |
| 101) | | | | |
| Hypromellose | 2.6 | 3.9 | 10.5 | 12 |
| (Methocel ™ E-5) | | | | |
| Ethyl Acrylate and | 4.8 | 7.2 | 19.3 | 22.2 |
| Methyl Methacrylate | | | | |
| Copolymer Disp. | | | | |
| (Eudragit ™ NM 30D) | | | | |
| Polysorbate 80 (Tween | 0.5 | 0.7 | 1.9 | 2.2 |
| 80HP-LQ-MH) | | | | |
| Eudragit ™ FS 30 D, | 6.4 | 9.7 | 25.8 | 29.6 |
| Aq. Dispersion, | | | | |
| [Poly(Methyl Acrylate- | | | | |
| CO-Methyl | | | | |
| Methacrylate-CO- | | | | |
| Methacrylic Acid)] | | | | |
| PlasACRYL ™ T20 | 0.6 | 1 | 2.6 | 3 |
| Talc, 194 Pharma M | 5.6 | 8.5 | 22.6 | 26 |
| Colloidal Silicon | 0.4 | 0.6 | 1.6 | 1.8 |
| Dioxide (Aerosil ™ 200 | | | | |
| Pharma) | | | | |
| Vacant HPMC Capsule | 1 unit | 1 unit | 1 unit | 1 unit |
| shell | (size3) | (size2) | (size0) | (size0) |
| Total | 108.5 | 163.3 | 354.3 | 407.3 |

TABLE 8
Examples of centanafadine extended-release capsules

| | 41.1 mg | 82.2 mg | 123.3 mg |
|---|---|---|---|
| | Capsules | Capsules | Capsules |
| | Quantity/ | Quantity/ | Quantity/ |
| Component | Capsule(mg) | Capsule(mg) | Capsule(mg) |
| Centanafadine | 41.1 | 82.2 | 123.3 |
| hydrochloride | | | |
| Mannitol (PEARLITOL ™ | 24.9 | 2 | 3 |
| 50C) | | | |
| Microcrystalline Cellulose | 28.9 | 18 | 27 |
| (Avicel ™ PH-301) | | | |
| Microcrystalline Cellulose | 0 | 8 | 11.9 |
| (Avicel ™ PH-101) | | | |
| Hypromellose (Methocel ™ | 1.9 | 3.8 | 5.6 |
| E-5) | | | |
| Ethyl Acrylate and Methyl | 3.5 | 7 | 10.5 |
| Methacrylate Copolymer | | | |
| Disp. (Eudragit ™ NM | | | |
| 30D)* | | | |
| Polysorbate 80 (Tween | 0.3 | 0.7 | 1 |
| 80HP-LQ-MH) | | | |
| Eudragit ™ FS 30 D, Aq. | 4.7 | 9.3 | 14 |
| Dispersion, [Poly(Methyl | | | |
| Acrylate-CO-Methyl | | | |
| Methacrylate-CO- | | | |
| Methacrylic Acid)]* | | | |
| PlasACRYL ™ T20* | 0.5 | 0.9 | 1.4 |
| Talc, 194 Pharma M | 4.3 | 8.3 | 12.4 |
| Colloidal Silicon Dioxide | 0.3 | 0.6 | 0.9 |
| (Aerosil ™ 200 Pharma) | | | |
| Vacant HPMC Capsule | 1 unit | 1 unit | 1 unit |
| shell | (size 4) | (size 3) | (size 1) |
| Total | 110 | 141 | 211 |

TABLE 9
Examples of centanafadine extended-release capsules

| | 164.4 mg | 246.6 mg | 328.8 mg |
|---|---|---|---|
| | Capsules | Capsules | Capsules |
| | Quantity/ | Quantity/ | Quantity/ |
| Component | Capsule(mg) | Capsule(mg) | Capsule(mg) |
| Centanafadine | 164.4 | 246.6 | 328.8 |
| hydrochloride | | | |
| Mannitol (PEARLITOL ™ | 4 | 6 | 8 |
| 50C) | | | |
| Microcrystalline Cellulose | 36.2 | 54.2 | 72.2 |
| (Avicel ™ PH-301) | | | |
| Microcrystalline Cellulose | 15.9 | 23.9 | 31.8 |
| (Avicel ™ PH-101) | | | |
| Hypromellose | 7.5 | 11.3 | 15 |
| (Methocel ™ E-5) | | | |
| Ethyl Acrylate and Methyl | 13.9 | 20.9 | 27.9 |
| Methacrylate Copolymer | | | |
| Disp. (Eudragit ™ NM | | | |
| 30D)* | | | |
| Polysorbate 80 (Tween | 1.4 | 2.1 | 2.8 |
| 80HP-LQ-MH) | | | |
| Eudragit ™ FS 30 D, Aq. | 18.6 | 27.9 | 37.2 |
| Dispersion, [Poly(Methyl | | | |
| Acrylate-CO-Methyl | | | |
| Methacrylate-CO- | | | |
| Methacrylic Acid)]* | | | |
| PlasACRYL ™ T20* | 1.9 | 2.8 | 3.7 |
| Talc, 194 Pharma M | 16.2 | 24.8 | 33.1 |
| Colloidal Silicon Dioxide | 1.1 | 1.7 | 2.3 |
| (Aerosil ™ 200 Pharma) | | | |
| Vacant HPMC Capsule | 1 unit | 1 unit | 1 unit |
| shell | (size 1) | (size 0EL) | (size 00) |
| Total | 282 | 422 | 563 |

*net solid content

TABLE 10
Bead type composition of centanafadine extended-release capsules

| | | All weights below in Salt Form | | | | | | | | | | | |
|---|---|---|---|---|---|---|---|---|---|---|---|---|---|
| | | | | | SR Beads (15% | | | | DR Beads (20% | | | | |
| Total | | IR Beads | | | WG) | | | | WG) | | | | Total |
| Strength | | | | Fill | | | Fill | | | | Fill | | Fill |
| (Salt) | | Strength | | weight | Strength | | weight | | Strength | | weight | | Weight |
| 41.1 | mg | 5 | mg | 50 mg | 18.05 | mg | 31 | mg | 18.05 | mg | 29 | mg | 110 mg |
| | | | | (10% DL) | | | | | | | | | |
| 82.2 | mg | 10 | mg | 20 mg | 36.1 | mg | 63 | mg | 36.1 | mg | 58.0 | mg | 141 mg |
| | | | | (50% DL) | | | | | | | | | |
| 123.3 | mg | 15 | mg | 30 mg | 54.15 | mg | 94 | mg | 54.15 | mg | 87 | mg | 211 mg |
| | | | | (50% DL) | | | | | | | | | |
| 164.4 | mg | 20 | mg | 40 mg | 72.2 | mg | 126 | mg | 72.2 | mg | 116 | mg | 282 mg |
| | | | | (50% DL) | | | | | | | | | |
| 246.6 | mg | 30 | mg | 60 mg | 108.3 | mg | 188 | mg | 108.3 | mg | 174 | mg | 422 mg |
| | | | | (50% DL) | | | | | | | | | |
| 328.8 | mg | 40 | mg | 80 mg | 144.4 | mg | 251 | mg | 144.4 | mg | 232 | mg | 563 mg |

(50% DL)

The plasma pharmacokinetics from a CTN HCl formulation having a dose of 164.4 mg can be as in Table 11.

TABLE 11
Plasma pharmacokinetics

| Parameter^{a} | Centanafadine HCl 164.4 mg |
|---|---|
| Cₘₐₓ (ng/mL) | About 500 to about 700, or 618, or 629, or 651 |
| C_{1 hr} (ng/mL) | About 200 to about 400, or 302, or 321, or 358 |
| C_{12 hr} (ng/mL) | About 150 to about 300, or 186, or 220, or 289 |
| C_{16 hr} (ng/mL) | About 50 to about 150, or 80.9, or 98.4, or 133 |
| tₘₐₓ (h) | About 4 to about 7.5, or 4.5, or 5, or 7 |
| AUC_{0-1 h} | About 150 to about 500, or 215 or 395 |
| (ng · h/mL) | |
| AUC_{0-24 h} | Aboutu 4500 to about 7500, or 5290, or 6570, or |
| (ng · h/mL) | 5716 |
| AUC_{0-inf} | About 5000 to about 8000, or 6240, or 7110 |
| (ng · h/mL) | |
| (n = 11) | |

^{a}Values are mean (SD) except for tmax which is median (minimum-maximum).

The plasma pharmacokinetics from a CTN HCl formulation having a dose of 164.4 mg or 328.8 mg can be as in Table 12.

TABLE 12
Plasma pharmacokinetics

| | Centanafadine HCl 164.4 mg | | | | Centanafadine HCl 328.8 mg | | | |
|---|---|---|---|---|---|---|---|---|
| | (n = 8) | | | | (n = 8) | | | |
| Parameter^{a} | First Day | | Fifth Day | | First Day | | Fifth Day | |
| Cₘₐₓ (ng/mL) | 651 | (252) | 709 | (300) | 1940 | (781) | 2230 | (577) |
| C_{1 hr} (ng/mL) | 358 | (104) | 427 | (161) | 640 | (369) | 718 | (240) |
| C_{12 hr} (ng/mL) | 289 | (79.5) | 276 | (153) | 750 | (325) | 646 | (241) |
| C_{16 hr} (ng/mL) | 133 | (43.2) | 103 | (61.5) | 306 | (182) | 211 | (64.2) |
| tₘₐₓ (h) | 7.00 | (0.5-12.00) | 5.50 | (1.00-8.00) | 4.00 | (1.00-8.00) | 5.00 | (3.00-8.00) |
| AUC_{0-24 h} | 6570 | (1960) | 6650 | (2780) | 16000 | (4710) | 18400 | (4310) |
| (ng · h/mL) | | | | | | | | |
| AUC_{0-inf} | 7110 | (2000) | | | 16700 | (4650) | | |
| (ng · h/mL) | | | | | | | | |

| | | | | | |
|---|---|---|---|---|---|
| Cₘₐₓ ratio^{b} | — | 1.13 | (0.78-1.75) | 1.31 | (0.73-1.64) |
| AUC ratio^{b} | — | 0.98 | (0.72-1.17) | 1.16 | (0.88-1.54) |

^{a}Values are mean (SD) except for tmax which shows median (minimum-maximum).
^{b}Parameter ratios, Fifth Day/First Day

When two active agents are administered, the therapeutically effective amount of each agent optionally can be below the amount needed for activity as monotherapy. Accordingly, a subthreshold amount (i.e., an amount below the level necessary for efficacy as monotherapy) can be considered therapeutically effective. Indeed, in certain circumstances, an advantage of administering different agents with different mechanisms of action and different side effect profiles can be to reduce the dosage and side effects of either or both agents.

### Combination Therapy

In some embodiments, the disclosure provides methods of using centanafadine or a pharmaceutically acceptable salt thereof for treating MDD or for alleviating, improving or relieving MDD symptoms, in combination with one or more other antidepressant(s), anti-psychotic agents, or cognitive enhancers. Examples of classes of antidepressants that can be used in combination with centanafadine or a pharmaceutically acceptable salt thereof include norepinephrine reuptake inhibitors, selective serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotropin releasing factor (CRF) antagonists, α-adrenoreceptor antagonists, benzodiazepines, anxiolytics and atypical antidepressants.

Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Suitable selective serotonin reuptake inhibitors include fluoxetine, citalopram, escitalopram, fluvoxamine, paroxetine, dapoxetine, vortioxetine, and sertraline.

Examples of monoamine oxidase inhibitors include isocarboxazid, phenelzine, and tranylcypromine. Suitable reversible inhibitors of monoamine oxidase include moclobemide.

Suitable serotonin and noradrenaline reuptake inhibitors of use in the present invention include venlafaxine, nefazodone, milnacipran, and duloxetine.

In some embodiments, the disclosure provides methods of treating MDD or for alleviating, improving or relieving MDD symptoms, in a patient in need thereof which comprise administering a therapeutically effective amount of centanafadine or a pharmaceutically acceptable salt thereof in combination with one or more natural remedies, or supplements, which are promoted by marketers as helping with depression such as, but not limited to: St. John's wort (herbal supplement), S-adenosylmethionine (SAMe), omega-3 fatty acids, saffron, 5-hydroxytryptophan (5-HTP), dehydroepiandrosterone (DHEA).

In some embodiments, the disclosure provides methods of treating MDD or for alleviating, improving or relieving MDD symptoms, in a patient in need thereof which comprises administering a therapeutically effective amount of centanafadine or a pharmaceutically acceptable salt thereof in combination with one or more other antidepressant(s), anti-psychotic agents, or cognitive enhancers, wherein the combination is a single formulation, or multiple combination that are co-administered within a specific period, and for a duration of time. In some embodiments, the disclosure provides methods for treating MDD or for alleviating, improving or relieving MDD symptoms, in a patient in need thereof which comprises administering to said patient, a) a therapeutically effective amount of (1R,5S)-1-(naphthalen-2-yl)-3-azabicyclo[3.1.0]hexane or a pharmaceutically acceptable salt thereof, and b) one or more antidepressants, optionally selected from selective serotonin reuptake inhibitors (SSRIs) and/or serotonin-norepinephrine reuptake inhibitors (SNRTs), optionally selected from SSRTs, and further optionally selected from the group of fluoxetine, sertraline, paroxetine, citalopram, escitalopram, fluvoxamine, vilazodone, venlafaxine, desvenlafaxine, dapoxetine, vortioxetine, venlafaxine and duloxetine, wherein (1R,5S)-1-(naphthalen-2-yl)-3-azabicyclo[3.1.0]hexane or a pharmaceutically acceptable salt thereof is administered in an extended-release dosage form, as described herein.

In some embodiments, the one or more SSRIs is selected from fluoxetine, sertraline, paroxetine, fluvoxamine, citalopram, escitalopram, and vilazodone.

In some embodiments, the one or more SSRIs is selected from fluoxetine, sertraline, paroxetine, citalopram, and escitalopram.

In some embodiments, the one or more SSRIs is selected from fluoxetine and fluvoxamine.

In some embodiments, the SSRI is fluvoxamine.

In some embodiments, the SSRI is escitalopram.

In some embodiments, the one or more SNRIs is selected from venlafaxine, desvenlafaxine, duloxetine, levomilnacipran and reboxetine.

In some embodiments, the antidepressant comprises escitalopram.

In some embodiments, the antidepressant comprises citalopram.

A pharmaceutical combination therapy composition can include therapeutically effective amounts of CTN or a pharmaceutically acceptable salt thereof, as noted above, and a therapeutically effective amount of the additional therapeutic agent. In embodiments wherein the method comprises administering CTN or a pharmaceutically acceptable salt thereof in combination with one or more antidepressants (e.g., SSRIs and/or one or more SNRIs), the additional antidepressant is administered in a suitable amount. By way of example, therapeutic doses of antidepressants are shown in Table 13.

TABLE 13
Listing of Therapeutic Doses of other antidepressants

| Antidepressant | Therapeutic dose (daily mg) |
|---|---|
| Paroxetine | 20 to 60 (strengths 10, 20, 30 or 40) |
| Sertraline | 50 to 200 (strengths 25, 50, or 100) |
| Fluvoxamine | 100 to 300 (strengths 25, 50, or 100) |
| Fluoxetine | 20 to 80 (strengths 10, 20, or 40) |
| Citalopram | 20 to 40 (strengths 10, 20, or 40) |
| Escitalopram | 20 to 40 (strengths 5, 10, 20) |
| Venlafaxine (ER) | 37.5 to 300 or 25, 50, 75, 100, 37.5, 150, or 225 |
| Vilazodone | 10 to 40 (strengths 10, 20, or 40) |
| | Vilazodone Package Insert/Prescribing Information - |
| | Drugs.com |
| Desvenlafaxine | 50 to 400, (strengths 25, 50, or 100) |
| | 50 orally once a day, with or without food. |
| | Maximum dose: 400 mg/day |
| | Desvenlafaxine Dosage Guide + Max Dose, |
| | Adjustments - Drugs.com |
| Duloxetine | 40-60 to 120 (strengths 20, 30, 40, or 60) |
| | Initial dose: 20 mg to 30 mg orally 2 times a day |
| | Maintenance dose: 60 mg per day, given either once a |
| | day OR 30 mg orally 2 times a day |
| | Maximum dose: 120 mg/day |
| | Duloxetine Dosage Guide + Max Dose, Adjustments - |
| | Drugs.com |
| Levomilnacipran | 40 to 120 (strengths 20, 40, 80, or 120) |
| | Initial dose: 20 mg orally once a day for 2 days, then |
| | increase to 40 mg orally once a day |
| | Maintenance dose: 40 to 120 mg orally once a day |
| | Maximum dose: 120 mg/day |
| | Levomilnacipran Dosage Guide + Max Dose, |
| | Adjustments - Drugs.com |
| Milnacipran | 12.5 to 200 (strengths 12.5, 25, 50 or 100) |
| | The recommended dose of Milnacipran HCl tablets is |
| | 100 mg/day (50 mg twice daily). |
| | Milnacipran Package Insert/Prescribing Information - |
| | Drugs.com |
| Atomoxetine | 40 to 100 (strengths 10, 18, 25, 40, 60, 80 or 100) |
| | Initial Dose: 40 mg/day orally. |
| | Maintenance Dose: Increase dose to 80 mg/day orally |
| | after a minimum of 3 days at the initial dose. |
| | Maximum Dose: After 2 to 4 additional weeks, the |
| | dose may be increased up to 100 mg/day in patients |
| | who have not achieved an optimal response. |
| | Atomoxetine Dosage Guide + Max Dose, |
| | Adjustments - Drugs.com |
| Dapoxetine | 30-60, (strengths 30 or 60) |
| Vortioxetine | 10 to 20, (strengths 5, 10 or 20) |
| Venlafaxine | 75 to 375, (strengths 25, 37.5, 50, 75 or 100) |
| Reboxetine | 8 to 12 (strengths 4 or 10) |

In some embodiments, a low dose of the antidepressant is initially administered to the patient and then is titrated up to the recommended therapeutic dose over a period of time.

In some embodiments, the antidepressant is escitalopram, wherein the amount of escitalopram or a pharmaceutically acceptable salt thereof (such as escitalopram oxalate or escitalopram hydrobromide) administered is in a range of about 2.5, 4, 5, 10, 15 or 20 mg daily. In some embodiments, the amount of escitalopram administered is about 10 mg daily. In some embodiments, the amount of escitalopram administered is about 20 mg daily.

In embodiments comprising combination therapy, CTN or a pharmaceutically acceptable salt thereof can be administered with the additional antidepressant agent (e.g., SSRIs and/or SNRIs) simultaneously (e.g., concurrently) or at separate intervals (e.g. sequentially). When administered simultaneously, CTN or a pharmaceutically acceptable salt thereof and the additional therapeutic agent can be incorporated into a single pharmaceutical composition, e.g., a pharmaceutical combination therapy composition. Alternatively, two separate compositions, i.e., one containing CTN or a pharmaceutically acceptable salt thereof and the other containing the additional therapeutic agent, can be administered simultaneously.

The dosage forms comprising combination therapy CTN or a pharmaceutically acceptable salt thereof and/or escitalopram may be formulated to provide modified release of the CTN or a pharmaceutically acceptable salt thereof and immediate or modified release of the escitalopram or a pharmaceutically acceptable salt thereof. The modified release profiles for CTN or a pharmaceutically acceptable salt thereof, escitalopram or a pharmaceutically acceptable salt thereof, or both can be achieved by a combination of sustained and pulsatile formulations.

Pulsatile release profiles can be achieved with dosage forms that are closed, such as sealed capsules or tablets, which contain two or more drug-containing dosage units. The dosage form can include one, two, three, or four or more types of dosage units, each having a different drug release profile. Each dosage unit can provide multi-phase release of the CTN or a pharmaceutically acceptable salt thereof and/or escitalopram or a pharmaceutically acceptable salt thereof.

The combination therapy can comprise administering CTN as described herein, in combination with counseling and/or behavioral therapy. The combination therapy can comprise administering CTN as described herein, in combination with digital therapy and/or digital apps. The combination therapy can comprise administering CTN and escitalopram as described herein, in combination with counseling and/or behavioral therapy. The combination therapy can comprise administering CTN and escitalopram as described herein, in combination with digital therapy and/or digital apps.

When administered separately, therapeutically effective amounts of compositions containing CTN or a pharmaceutically acceptable salt thereof and the additional therapeutic agent can be administered on a different schedule. One may be administered before the other as long as the time between the two administrations falls within a therapeutically effective interval. A therapeutically effective interval is a period of time beginning when one of either (a) CTN or a pharmaceutically acceptable salt thereof, or (b) the additional therapeutic agent is administered to a human and ending at the limit of the beneficial effect in the treating MDD or in the alleviating, improving or relieving MDD symptoms, of the combination of (a) and (b). The methods of administration of the CTN or a pharmaceutically acceptable salt thereof and the additional therapeutic agent may vary. Thus, either agent or both agents may be administered rectally, topically, orally, sublingually, or parenterally.

### EMBODIMENTS

1. A method for treating major depressive disorder or improving, alleviating, or relieving major depressive disorder symptoms in a human patient in need thereof, comprising administering to said patient a therapeutically effective amount of centanafadine or a pharmaceutically acceptable salt thereof, wherein centanafadine or a pharmaceutically acceptable salt thereof is administered in an extended-release dosage form comprising a plurality of centanafadine beads.

2. The method of 1, wherein the plurality of centanafadine beads each comprises a core particle comprising centanafadine or a pharmaceutically acceptable salt thereof, and an excipient, and wherein the plurality of centanafadine beads comprises a mixture of one or more immediate release beads, one or more sustained release beads, and one or more delayed release beads.

3. The method of 2, wherein the one or more sustained release beads comprises a sustained release coating comprising one or more materials selected from an alkylcellulose, acrylic acid polymer, a methacrylic acid polymer, an acrylic acid copolymer, a methacrylic acid copolymer, and a cellulose ether.

4. The method of 2 or 3, wherein the one or more delayed release beads comprises a delayed release coating comprising one or more materials selected from amylose acetate phthalate, cellulose acetate phthalate, cellulose acetate succinate, cellulose acetate trimellitate, carboxymethyl ethylcellulose, co-polymerized methacrylic acid/methacrylic acid methyl esters, co-polymerized methacrylic acid/methyl methacrylate, co-polymerized methylacrylate/methyl methacrylate/methacrylic acid, hydroxypropyl methylcellulose acetate succinate, hydroxypropyl methylcellulose phthalate, polyvinyl acetate phthalate, styrene maleic acid copolymer, and styrene vinylpyridine copolymer.

5. The method of any one of 2 to 4, wherein the mixture of the one or more immediate release beads, one or more sustained release beads, and one or more delayed release beads is in a ratio in a range of about 0.1-1:1-20:1-20 parts.

6. The method of any one of 1 to 5, wherein the extended-release dosage form is a daily oral dosage form.

7. The method of 6, wherein the dosage form is a capsule.

8. The method of any one of 1 to 7, wherein the pharmaceutically acceptable salt of centanafadine is the hydrochloride salt.

9. The method of any one of 1 to 8, wherein the amount of the centanafadine or a pharmaceutically acceptable salt thereof administered is in a range of about 10 mg to about 750 mg daily.

10. The method of 9, wherein the amount of the centanafadine or a pharmaceutically acceptable salt thereof administered is in a range of about 35 mg to about 400 mg daily.

11. A method for treating major depressive disorder or improving, alleviating, or relieving major depressive disorder symptoms in a patient in need thereof which comprises administering to said patient, a therapeutically effective amount of centanafadine or a pharmaceutically acceptable salt thereof, as adjunct to one or more antidepressants.

12. The method of claim 11, wherein the one or more antidepressants is/are selected from selective serotonin reuptake inhibitors, serotonin-norepinephrine reuptake inhibitors, benzodiazepines, anxiolytics, noradrenergic and specific serotonergic antidepressants, tricyclic antidepressants and monoamine oxidase inhibitors.

13. The method of 11, wherein the one or more antidepressants is/are selective serotonin reuptake inhibitors.

14. The method of 13, wherein the selective serotonin reuptake inhibitor comprises escitalopram.

15. The method of any of 11-14, wherein centanafadine or a pharmaceutically acceptable salt thereof, is administered in an extended-release dosage form comprising a plurality of centanafadine beads.

16. The method of 15, wherein the plurality of centanafadine beads each comprises a core particle comprising centanafadine or a pharmaceutically acceptable salt thereof, and an excipient, and wherein the plurality of centanafadine beads comprises a mixture of one or more immediate release beads, one or more sustained release beads, and one or more delayed release beads.

17. The method of any of 16, wherein the one or more sustained release beads comprises a sustained release coating comprising one or more materials selected from an alkylcellulose, acrylic acid polymer, a methacrylic acid polymer, an acrylic acid copolymer, a methacrylic acid copolymer, and a cellulose ether.

18. The method of 16 or 17, wherein the one or more delayed release beads comprises a delayed release coating comprising one or more materials selected from amylose acetate phthalate, cellulose acetate phthalate, cellulose acetate succinate, cellulose acetate trimellitate, carboxymethyl ethylcellulose, co-polymerized methacrylic acid/methacrylic acid methyl esters, co-polymerized methacrylic acid/methyl methacrylate, co-polymerized methylacrylate/methyl methacrylate/methacrylic acid, hydroxypropyl methylcellulose acetate succinate, hydroxypropyl methylcellulose phthalate, polyvinyl acetate phthalate, styrene maleic acid copolymer, and styrene vinylpyridine copolymer.

19. The method of any of 16 to 18, wherein the mixture of the one or more immediate release beads, one or more sustained release beads, and one or more delayed release beads is at a ratio in a range of about 0.1-1:1-20:1-20 parts.

20. The method of any of 11 to 19, wherein the amount of the centanafadine or a pharmaceutically acceptable salt thereof is administered in a range of about 10 mg to about 750 mg daily as adjunct to escitalopram or a pharmaceutically acceptable salt thereof in amount of about 2.5 to about 20 mg daily.

21. The method of 20, wherein the amount of the centanafadine or a pharmaceutically acceptable salt thereof administered is in a range of about 35 mg to about 400 mg daily as adjunct to escitalopram or a pharmaceutically acceptable salt thereof in amount of about 10 to about 20 mg daily.

22. A method for treating major depressive disorder or improving, alleviating, or relieving major depressive disorder symptoms in a patient in need thereof which comprises administering to said patient, a therapeutically effective amount of centanafadine or a pharmaceutically acceptable salt thereof, as adjunct to a selective serotonin reuptake inhibitor; wherein centanafadine or a pharmaceutically acceptable salt thereof, is administered in an extended-release dosage form comprising a plurality of centanafadine beads; wherein the plurality of centanafadine beads each comprises a core particle comprising centanafadine or a pharmaceutically acceptable salt thereof, and an excipient;
wherein the plurality of centanafadine beads comprises a mixture of one or more immediate release beads, one or more sustained release beads, and one or more delayed release beads; wherein the mixture of the one or more immediate release beads, one or more sustained release beads, and one or more delayed release beads is at a ratio in a range of about 0.1-1:1-20:1-20 parts; wherein centanafadine or a pharmaceutically acceptable salt thereof is administered in a range of about 35 mg to about 400 mg daily; wherein the selective serotonin reuptake inhibitor is escitalopram a pharmaceutically acceptable salt thereof; and wherein escitalopram or a pharmaceutically acceptable salt thereof is administered in a range of about 10 mg to about 20 mg daily.

23. The method of 22, wherein cetanafadine is administered as the hydrochloride salt and escitalopram is administered as the oxalate salt.

24. The method of 22 or 23, wherein the mixture of the one or more immediate release beads, one or more sustained release beads, and one or more delayed release beads is at a ratio in a range of about 1:3.6:3.6 parts.

25. The method of any one of 1 to 24, wherein centanafadine hydrochloride is administered in an amount of 328.4 mg daily and escitalopram oxalate is administered in an amount of 10 mg daily.

26. The method of any one of 1 to 25, wherein centanafadine hydrochloride is administered in an amount of 328.8 mg daily and escitalopram oxalate is administered in an amount of 20 mg daily.

### EXAMPLES

The following examples are provided for illustration and are not intended to limit the scope of the invention. In the examples below, centanafadine was formulated and administered as centanafadine hydrochloride, unless otherwise indicated.

In the examples below, escitalopram is administered according to LEXAPRO™ (escitalopram) US Prescribing Information. Allergan USA, Inc.; Madison, NJ; August 2020.

The phase 2 clinical trial described herein is a multicenter, randomized, double-blind, placebo-controlled, parallel-arm trial to assess the efficacy, safety, and tolerability of centanafadine extended-release capsules as monotherapy or as adjunct to SSRI in adult subjects with major depressive disorder. Therefore, the clinical trial has several objectives.

An objective of the trail is to assess the efficacy of centanafadine QD XR at 328.8 mg as monotherapy in adults with MDD.

Another objective of the trail is to assess the efficacy of centanafadine QD XR at 328.8 mg as adjunct to SSRI (escitalopram) in adults with MDD.

A further objective of the trail is to assess the safety and tolerability of centanafadine QD XR at 328.8 mg as monotherapy in adults with MDD.

Another objective of the trail is to assess the safety and tolerability of centanafadine QD XR at 328.8 mg as adjunct to SSRI (escitalopram) in adults with MDD.

Eligible subjects are randomized 1:1:1:1 at baseline to 1 of 4 treatment arms, as follows:
Treatment Arm 1: 2 centanafadine QD XR 164.4 mg oral capsules+1 placebo for escitalopram tablet;
Treatment Arm 2: 2 centanafadine QD XR 164.4 mg oral capsules+1 escitalopram tablet;
Treatment Arm 3: 1 escitalopram tablet+2 placebo for centanafadine QD XR capsules;
Treatment Arm 4: 2 placebo for centanafadine QD XR capsules+1 placebo for escitalopram tablet.
Treatment Arm 1: Eligible subjects receive centanafadine QD XR 328.8 mg and escitalopram matching placebo for 6 weeks (monotherapy). Treatment Arm 2: Eligible subjects receive centanafadine QD XR 328.8 mg and escitalopram, for 6 weeks (adjunct therapy).
Treatment Arm 3: Eligible subjects receive matching placebo for centanafadine QD XR 328.8 mg and escitalopram, for 6 weeks. Treatment Arm 4: Eligible subjects receive matching placebo for centanafadine QD XR 328.8 mg and matching placebo for escitalopram, for 6 weeks.

Eligible subjects assigned to receive centanafadine QD XR (i.e., Treatment Arms 1 or 2) receive 328.8 mg centanafadine (i.e., 2 centanafadine QD XR 164.4 mg oral capsules) starting on Day 1.

Eligible subjects assigned to receive escitalopram (i.e., Treatment Arms 2 or 3) receive a 10 mg QD dose at Day 1. At Week 1, dosing increases to 20 mg QD for the remainder of subject participation in the trial.

Subjects who cannot tolerate the daily dose administered any time during the 6-week treatment period or who require changes to medication to treat their depression are discontinued from the study. Dose reductions are not permitted.

### Example 1

Treatment of MDD Patients with Centanafadine Monotherapy

A Phase 2, multicenter, randomized, double-blind, placebo-controlled trial is conducted to assess the efficacy, safety, and tolerability of centanafadine as monotherapy in adult subjects with MDD. Subjects with major depression who meet inclusion and exclusion criteria are randomized 1:1 (84 subjects per treatment arm) at base line to receive 6 weeks of treatment with centanafadine extended-release (XR) capsules (328.8 mg centanafadine hydrochloride once daily) monotherapy, or placebo. The trial consists of a screening period of up to 28 days, a baseline visit, a 6-week double-blind treatment period, and a 7-day safety follow-up period. The trial is conducted on an outpatient basis.

Inclusion criteria are: Age 18-65 years old who have a current primary diagnosis of MDD, single or recurrent episode, without psychotic features and do not meet criteria for a mixed-features subtype, and are in a current major depressive episode (MDE) as defined by DSM-5 criteria and confirmed by the Mini International Neuropsychiatric Interview (MINI). The MINI is a widely used structured diagnostic interview instrument developed for DSM-5 psychiatric disorders. All subjects have HAM-D17 score of ≥18 at screening and baseline, BMI of 18 to 40 kg/m², inclusive, and a rating of 4 or higher on the CGI-S at screening and baseline. Other inclusion criteria include: Subjects who have reported inadequate response to at least 1 but no more than 3 treatments for depression in their current MDE. Inadequate response is defined as <50% reduction in depression symptom severity following adequate dose and duration (i.e., at least 6 weeks at a minimum therapeutic dose or higher) according to the MGHATRQ performed at screening. Subjects who, in the opinion of the investigator, can safely wash out of current antidepressants.

Exclusion criteria are: current pregnancy and/or breast-feeding, or have intention to become pregnant during a study; a history with current depressive episode for longer than 2 years; meeting to DSM-5 criteria for personality disorder, substance use disorder, or a lifetime or current comorbid psychiatric disorder that either would be likely to require treatment with prohibited concomitant medications or psychotherapy in this trial, or to confound efficacy or safety assessments (for example, experiencing hallucinations, delusions, or any psychotic symptomatology), allergic responses to the medication, or a history of dermatologic adverse reactions or anaphylaxis secondary to drug exposure; having epilepsy, a history of epilepsy, or a history of seizure; clinically significant current suicidal intent; significant cardiac, medical or progressive neurological or medical illness; facial pain or trigeminal neuralgia; having test and ECG results at screening: 1) Platelets <75,000/mm³, 2) Hemoglobin <9 g/dL 3) Neutrophils, absolute <1000/mm³; 4) AST>2× upper limit of normal; 5) ALT>2× upper limit of normal; 6) Creatinine ≥2 mg/dL; 7) HbA1c≥7%; 8) QTcF≥450 msec for males or ≥470 msec for females; 9) Abnormal free T4, unless discussed with and approved by the medical monitor. Other exclusion criteria include having a lifetime history of treatment with ketamine, arketamine, or esketamine; having been treated with psychopharmacological augmentation for depression (e.g., lithium, triiodothyronine, or antipsychotics added to ADT, multiple ADTs) in the current MDE; have started individual or group psychotherapy within the past 3 months prior to screening or having been treated with electroconvulsive therapy or neuromodulation devices (e.g., transcranial magnetic stimulation, vagus nerve stimulation, or transcranial direct current stimulation) for depression, or a history of non-compliance.

All subjects agree to discontinue all prohibited medications (i.e., various antidepressants, anticonvulsants, mood stabilizer, benzodiazepines, hypnotics, stimulants, opioid analgesics, nutritional supplements and non-prescription herbal preparations with central nervous system effects) during the screening period, in order to meet the protocol-specified washout periods, and during the trial. Subjects on an antidepressant at the time of screening undergo a washout prior to baseline. The required washout period depends on the type of antidepressant. For example, for SSRIs (e.g., sertraline), monoamine oxidase inhibitors (e.g., selegiline), serotonin-norepinephrine reuptake inhibitors (e.g., venlafaxine, duloxetine), norepinephrine-dopamine reuptake inhibitors (e.g., bupropion), or other antidepressants (e.g., vortioxetine, vilazodone), the required washout period is 7 days. For fluoxetine or fluvoxamine, the required washout period is 28 days. A drug screen is performed at screening and baseline (Day-1). The screening period is up to 4 weeks (up to 28 days).

All subjects receive either centanafadine XR capsules as monotherapy, or matching placebo, for 6 weeks. Subjects assigned to receive centanafadine once-daily XR (i.e., receive 328.8 mg (2 centanafadine QD XR 164.4 mg oral capsules) starting on Day 1. All doses of centanafadine XR, and matching placebo are to be taken orally with approximately 240 mL of water, in the morning upon waking, at approximately the same time each day. The dose is not to be taken following a high-fat meal. Subjects are instructed not to take more than 1 dose in 1 day.

Subjects who do not tolerate the daily dose administered any time during the 6-week treatment period or who require changes to medication to treat their depression are discontinued from the study. Efficacy assessments are made at screening and/or baseline (day-1), and then at weeks 1, 2, 4, and 6. After a 6-week double-blind treatment period, the subjects undergo a safety follow-up period of 7 (+2) days post Week-6/early termination (ET) visit.

### Primary Efficacy Assessment

The primary efficacy of centanafadine QD XR as a monotherapy in adults is assessed by evaluation of the change from baseline in MADRS Total score at Week 6. For analysis of the double-blind treatment phase data, baseline is defined as the last available measurement prior to the first dose of double-blind centanafadine QD XR.

The MADRS is a 10-item scale for the evaluation of depressive symptoms. Each MADRS item is rated on a 0 to 6 scale with zero being the “best” rating and 6 being the “worst” rating. Higher MADRS scores indicate higher levels of depressive symptoms. The MADRS Total score is the sum of ratings for all 10 items; therefore, possible total scores range from 0 to 60. The MADRS Total score will be unevaluable if less than 8 of the 10 items are recorded. If 8 or 9 of the 10 items are recorded, the MADRS Total score will be the mean of the recorded items multiplied by 10 and then rounded to the first decimal place.

### Secondary Efficacy Assessment

Other efficacy assessed in this study include: 1) change from baseline in MADRS Total score at Weeks 1, 2, and 4; 2) change from baseline in Clinical Global Impression—Severity (CGI-S) score; 3) Clinical Global Impression—Change (CGI-C) score; 4) Change from baseline in Patient Global Impression—Severity (PGI-S) score; 5) Patient Global Impression—Change (PGI-C) score; 6) Change from baseline in Symptoms of Major Depressive Disorder Scale (SMDDS) Total score; 7) Change from screening in 36-item Short-Form Health Survey Version 2 (SF-36v2); 8) Change from screening in Apathy Evaluation Scale—Self-rated (AES-S) score; 9) MADRS Response Rate (where response is defined as 50% reduction in MADRS Total score from baseline at scheduled visits); and 10) MADRS Remission Rate (where remission is defined as MADRS Total score ≤10 at scheduled visits). Exploratory efficacy assessed in this study also include change from baseline in MADRS anhedonia factor.

### Clinical Global Impression-Severity (CGI-S)

The CGI-S is a standardized clinician-administered global rating scale that measures disease severity on a 7-point scale, where a higher score represents a higher severity of disease. Response choices are 1=normal, not at all ill; 2=borderline ill; 3=mildly ill; 4=moderately ill; 5=markedly ill; 6=severely ill; and 7=among the most extremely ill patients. Eligible subjects have a score of 4 or greater on the CGI-S at screening and baseline.

### Clinical Global Impression—Change (CGI-C)

The CGI-C is a single-item, 7-point scale that requires the clinician to assess how much the subject's illness has improved or worsened relative to the beginning of treatment, rated as: 1=very much improved; 2=much improved; 3=minimally improved; 4=no change; 5=minimally worse; 6=much worse; or 7=very much worse.

### Patient Global Impression—Severity (PGI-S)

The PGI-S is a single-item report of the subject's severity of symptoms. Subjects are asked “Taking into account all of your symptoms, how severe is your depression at this time?” Scores range from 1 “no symptoms” to 7 “very severe.”

### Patient Global Impression—Change (PGI-C)

The PGI-C is a 7-point single-item self-report scale depicting a patient's rating of overall change in their condition since starting trial medication. Subjects answer the following question: “Since starting study medication, how much have your symptoms of depression changed?” Scores range from 1 “very much improved” to 7 “very much worse.”

### 36-Item Short-Form Health Survey Version 2 (SF-36v2)

The SF-36v2 is a subject-reported questionnaire with a standard recall period of 4 weeks which measures generic health-related quality of life on 2 broad domains, physical and mental composites, across 8 health domain scales: physical functioning, pain, role physical, general health, vitality/fatigue, social functioning role emotional, and mental health. The SF-36v2 uses norm-based scoring to generate scores on a scale of 0 to 100, where lower scores on the physical component summary and mental component summary represents a lower health-related quality of life and a score of 50 references the normal US population. In addition to the composite scores and the individual health domain scores, the SF-36v2 provides a risk for depression score and the SF-36v2 health utility index on a scale form 0.0 (worst measured health state) to 1.0 (best measured health state).

### Symptoms of Major Depressive Disorder Scale (SMDDS)

The SMDDS is a 16-item patient-reported outcome (PRO) questionnaire that was developed following guidelines suggested by the Food and Drug Administration (FDA) PRO Guidance (2009) and the Good Research Practices Task Force Reports from the International Society for Pharmacoeconomics and Outcomes Research for development and validation of a PRO for medical product evaluation. It is designed to assess subject-perceived severity of MDD symptoms over the past 7 days in adults being treated for MDD in an ambulatory setting. The SMDDS is comprised of 9 domains of MDD symptoms: negative emotions/mood (4 items), anxiety (2), low energy (1), cognition (2), sleep disturbance (1), self-harm/suicide (1), sense-of-self (1), and eating behavior (2 items scored as a single item). The SMDDS was qualified in 2017 by the FDA to assess overall symptoms of MDD in a trial setting.

### Apathy Evaluation Scale (AES-S)

The Apathy Evaluation Scale was developed to assess apathy as a “psychological dimension defined by simultaneous deficits in overt behavioral, cognitive, and emotional concomitants of goal-directed behavior.” The self-rated (AES-S) version has 18 items and uses a response scale of “Not at all,” “Slightly,” “Somewhat,” and “A lot.” The AES-S asks the respondent to answer based on “thoughts, feelings, and activity in the past 4 weeks.” Items are categorized into domains of goal-directed behavior, goal-related conditions, or goal-related emotional responses; scores range from 18 to 72, and a higher score indicates a higher level of apathy.

### Safety Assessment

Safety is assessed by standard measurements including treatment-emergent adverse events (TEAEs), adverse events of special interest (AESIs [rash]), clinical laboratory tests (chemistry, hematology, and urinalysis), physical examinations, vital sign measurements, and 12-lead ECGs. The Study Medication Withdrawal Questionnaire (SMWQ), Generalized Anxiety Disorder Assessment (GAD-7), and the Columbia-Suicide Severity Rating Scale (C-SSRS) is also used to assess safety. Abuse potential is assessed through the active monitoring of events subject to additional monitoring (ESAMs); e.g., AEs related to abuse potential and AEs involving a medication handling irregularity (MHI).

### Results

CTN significantly reduces all primary efficacy measures (i.e., MADRS Total score) in patients who remain after 6-week double-blind treatment period, whereas no significant reductions are observed in the placebo group. In addition, significant reductions in patient's CGI-S scores, CGI-C scores, PGI-S scores, and the PGI-C scores from baseline are also observed in patients receiving CTN whereas no significant reductions are observed in the placebo group. Increased SF-36v2 general scores from baseline as well as reduced risk for depression are also observed in the patients receiving CTN.

Centanafadine (CTN) is effective as monotherapy in patients with MDD by clinically significant improvements.

### Example 2

### Centanafadine as an Adjunct Therapy for the Treatment of MDD

A Phase 2, multicenter, randomized, double-blind, placebo-controlled trial is conducted to assess the efficacy, safety, and tolerability of centanafadine XR capsules as adjunct to SSRI (c) in adult subjects with MDD. Subjects with major depression who meet inclusion and exclusion criteria are randomized 1:1:1 (84 per treatment arm) at base line to receive 6 weeks of treatment with centanafadine XR capsules (328.8 mg centanafadine hydrochloride once daily)+escitalopram, escitalopram monotherapy (i.e., escitalopram once-daily (QD) dose), or placebo. The trial consists of a screening period of up to 28 days, a baseline visit, a 6-week double-blind treatment period, and a 7-day safety follow-up period. The trial is conducted on an outpatient basis.

Inclusion and Exclusion criteria are the same as Example 1. In addition, subjects who are taking escitalopram or citalopram at the time of screening, have been exposed to escitalopram or citalopram in their current MDE, or have reported an inadequate response to an adequate dose/duration of escitalopram or citalopram in the past, are also excluded.

All subjects agree to discontinue all prohibited medications during the screening period, in order to meet the protocol-specified washout periods, and during the trial. Subjects on an antidepressant at the time of screening undergo a washout prior to baseline. The required washout period depends on the type of antidepressant (see Example 1). A drug screen is performed at screening and baseline (Day-1). The screening period is up to 4 weeks (up to 28 days).

All subjects receive either escitalopram monotherapy, centanafadine QD XR capsules+escitalopram (combination therapy), or matching placebo, for 6 weeks.

Subjects assigned to receive centanafadine XR+escitalopram combination therapy receive 328.8 mg centanafadine (i.e., 2 centanafadine QD XR 164.4 mg oral capsules) and 1 escitalopram tablet starting on Day 1. For day 1, the escitalopram tablet is a 10 mg QD dose. At Week 1, dosing increases to 20 mg QD for the remainder of subject participation in the trial.

Subjects assigned to receive escitalopram monotherapy receive a 10 mg QD dose at Day 1. At Week 1, dosing increases to 20 mg QD for the remainder of subject participation in the trial. Dose reductions are not permitted. Subjects who do not tolerate the daily dose administered any time during the 6-week treatment period or who require changes to medication to treat their depression are discontinued from the study.

All doses of centanafadine QD XR, escitalopram, and matching placebo are to be taken orally with approximately 240 mL of water, in the morning upon waking, at approximately the same time each day. The dose is not to be taken following a high-fat meal. After a 6-week double-blind treatment period, the subjects undergo a safety follow-up period of 7 (+2) days post Week-6/early termination (ET) visit.

### Efficacy Assessment

The primary efficacy of centanafadine QD XR as adjunct to SSRI (escitalopram) in adults subjects with MDD is assessed by comparing the efficacy between centanafadine QD XR+escitalopram and escitalopram monotherapy or the change from baseline in MADRS Total score at Week 6. For analysis of the double-blind treatment phase data, baseline is defined as the last available measurement prior to the first dose of double-blind escitalopram, or centanafadine QD XR+centanafadine.

Other efficacy assessed in this study include: 1) change from baseline in MADRS Total score at Weeks 1, 2, and 4; 2) change from baseline in Clinical Global Impression—Severity (CGI-S) score; 3) Clinical Global Impression—Change (CGI-C) score; 4) Change from baseline in Patient Global Impression—Severity (PGI-S) score; 5) Patient Global Impression—Change (PGI-C) score; 6) Change from baseline in Symptoms of Major Depressive Disorder Scale (SMDDS) Total score; 7) Change from screening in 36-item Short-Form Health Survey Version 2 (SF-36v2); 8) Change from screening in Apathy Evaluation Scale—Self-rated (AES-S) score; 9) MADRS Response Rate (where response is defined as 50% reduction in MADRS Total score from baseline at scheduled visits); and 10) MADRS Remission Rate (where remission is defined as MADRS Total score ≤10 at scheduled visits). These efficacy endpoints are defined the same as in Example 1. Exploratory efficacy assessed in this study also include change from baseline in MADRS anhedonia factor.

### Safety Assessment

Safety is assessed by standard measurements including treatment-emergent adverse events (TEAEs), adverse events of special interest (AESIs [rash]), clinical laboratory tests (chemistry, hematology, and urinalysis), physical examinations, vital sign measurements, and 12-lead ECGs. The Study Medication Withdrawal Questionnaire (SMWQ), Generalized Anxiety Disorder Assessment (GAD-7), and the Columbia-Suicide Severity Rating Scale (C-SSRS) are also used to assess safety. Abuse potential is assessed through the active monitoring of events subject to additional monitoring (ESAMs); e.g., AEs related to abuse potential and AEs involving a medication handling irregularity (MHI).

The mean decrease of all primary efficacy measures (i.e., MADRS Total score) in the patients after 6 weeks of combination therapy is greater than the mean decrease in the patients after 6 weeks of escitalopram monotherapy. No significant reductions are observed in the placebo group. Centanafadine (CTN) is effective as an adjunct therapy to SSRI (escitalopram) in patients with MDD.

### Method of Statistical Analysis

The sample size calculation of the trial is based on evaluating the efficacy of centanafadine QD XR as adjunctive therapy to escitalopram in adult subjects with MDD. The primary comparison is centanafadine QD XR+escitalopram versus escitalopram monotherapy for the primary efficacy endpoint, change from baseline in MADRS Total score at Week 6.

### Primary Efficacy—Endpoint Analysis

The objective of the primary efficacy analysis is to compare the efficacy between centanafadine QD XR+escitalopram and escitalopram monotherapy for the change from baseline in MADRS Total score at Week 6.

The primary estimand defining the treatment effect of interest in the protocol uses the hypothetical strategy specified in the International Council for Harmonisation (ICH) E9 Addendum. The estimand, or target of estimation, following the hypothetical strategy is the treatment effect seen, had no withdrawals occurred. Subjects who withdraw from IMP treatment either could have lost their treatment effect, had the subjects not taken any other treatment after withdrawal, or could have their treatment effect been masked, had the subjects taken other treatment after withdrawal. This means that any observations taken after subjects stop IMP most likely do not contribute relevant information about the treatment effect of the drug. Due to this strategy, the last collected efficacy assessment after premature trial discontinuation is done only once at the ET Visit. Every effort is made to complete all of the ET evaluations prior to administering any additional medications for the treatment of MDD. In the case of terminal or lost to follow-up events, no ET evaluations is expected, and only scheduled assessments performed before such an event has occurred.

The primary estimand for this trial is defined by the following components: Target Population: Efficacy Sample; Endpoint: Change from baseline to Week 6 in the MADRS Total score; Intercurrent Events: Premature treatment discontinuation; Measure of Intervention Effect: Difference in endpoint means between 6-week centanafadine QD XR+escitalopram and escitalopram monotherapy.

The primary efficacy analysis is performed by fitting a MMRM analysis with an unstructured (UN) variance covariance structure in which the change from baseline in MADRS Total score (at Weeks 1, 2, 4, and 6) is the dependent variable based on the observed cases (OC) data set on Efficacy Sample. The model includes fixed class effect terms for treatment, study center, visit week, and an interaction term of treatment by visit week. The model also includes the interaction term of baseline values of MADRS Total score by visit week as covariates. The primary comparison between centanafadine QD XR+escitalopram combination therapy and escitalopram monotherapy at Week 6 in the double-blind treatment period is estimated as the difference between Least Squares means utilizing the computing software SAS procedure PROC MIXED.

### Other Efficacy Endpoint Analysis

Comparisons are performed for the following other efficacy endpoints. All of these are tested at nominal 0.05 level.
1) Change from baseline in MADRS Total score at Weeks 1, 2, and 4;
2) Change from baseline in CGI-S score at Weeks 1, 2, 4, and 6;
3) CGI-C score at Weeks 1, 2, 4, and 6;
4) Change from baseline in PGI-S score at Weeks 1, 2, 4, and 6;
5) PGI-C score at Weeks 1, 2, 4, and 6;
6) Change from baseline in SMDDS Total score at Day 4, Weeks 1, 2, 4, and 6;
7) Change from screening in SF-36v2 physical component summary score, mental component summary score, and subscales of the SF-36v2;
8) Change from screening in AES-S score;
9) MADRS Response Rate, where response is defined as 50% reduction in MADRS Total score, from baseline at Weeks 1, 2, 4, and 6; and
10) MADRS Remission Rate, where remission is defined as MADRS Total score ≤10, at Weeks 1, 2, 4, and 6

Endpoints (1), (2), (4), and (6) are evaluated using the same MMRM model described in the primary analysis. Endpoints (3) and (5) are evaluated by the CMH Row Mean Score Differ Test controlling for study center using LOCF dataset. Endpoints (7) and (8) are evaluated using analysis of covariance with baseline value as covariate and treatment and, in LOCF analysis, study center as main effects. Endpoints (9) and (10) are evaluated by the CMH General Association Test controlling for study center using LOCF dataset. The OC analysis are also conducted for endpoints (3), (5), and (7) to (10) but do not control for study center.

### Exploratory Efficacy Endpoint Analysis

Change from baseline and descriptive statistics are used for the MADRS anhedonia factor score (total score of MADRS Items 1, 2, 6, 7, and 8).

### Pharmacokinetic Assessments

Pharmacokinetic samples are collected at weeks 2, 4 and 6 (±2 days).

### Pharmacokinetic Blood Samples

Single sparse PK samples are taken at Weeks 2, 4, and 6. The PK samples are collected after administering efficacy assessments and other safety assessments (AE monitoring, vital signs measurements, ECGs, medication withdrawal scales, and suicidality assessment). At Week 2, one sample is taken either prior to the morning dose (trough) or after 4 hours postdose; the alternate timepoint is then taken at Week 4. At Week 6, subjects are instructed to take their dose prior to a morning clinic visit and have a PK sample taken concurrently with the serum chemistry sample. Visit times are scheduled as needed to achieve the different sample times.

Blood samples are collected using venipuncture (2 mL whole blood, dipotassium ethylenediaminetetraacetic acid [K₂EDTA]) and are processed into plasma to determine the concentrations of centanafadine and metabolite(s).

When vital signs or electrocardiograms (ECGs) are scheduled at the same nominal time as PK sample collections, vital signs are measured, and ECGs are performed before PK samples are collected.

The actual date and time of the PK sample collection and the date and time of IMP administration prior to PK sample collection is recorded in the eCRF.

### Clinical Laboratory Assessments

Clinical laboratory samples are collected at the time of screening and baseline (Day-1), weeks 1, 4, and 6, to perform clinical laboratory assessments described herein and are collected after efficacy assessments are completed if possible.

Subjects are not required to be fasting for screening laboratory assessments. Laboratory assessments are fasted for subsequent laboratory assessments. Subjects are instructed to be fasting for a minimum of 8 hours prior to the blood draws, if possible.

All screening test results are available and inclusionary prior to randomization. Results of these tests are reviewed by personnel prior to initiation of the administration of the IMP. Additional urine and blood samples can be collected for further evaluation of safety as warranted.

Urine or serum pregnancy testing for subjects of CBP are performed during the study. On suspicion of pregnancy, an unscheduled urine or serum pregnancy test can be performed. Positive urine pregnancy tests are validated with a serum pregnancy test. Results of the pregnancy test must be available prior to the administration of the IMP. The subjects are counseled on the risk of pregnancy while participating in a clinical trial.

A drug screen is performed at screening and baseline (Day-1); additional assessments can be conducted at any time at the discretion of the investigator. Subjects with a validated positive drug screen for use of prohibited medications at screening are required to undergo a washout period. Subjects who have a validated positive drug screen for use of prohibited medications at baseline are considered screen failures and will not be permitted to rescreen.

Subjects are monitored for potentially clinically significant laboratory values.

### Other Safety Data

Medication withdrawal symptoms assessed by SMWQ total scores at the scheduled visit(s) are summarized by treatment arm by descriptive statistics.

Anxiety assessed by the GAD-7 at the scheduled visit(s) are summarized by treatment arm by descriptive statistics.

Suicidality monitored during the trial using the C-SSRS are summarized by treatment arm by descriptive statistics.

### Other Analyses

### Pharmacokinetic Analysis

Sparse PK samples are evaluated to verify compliance with dosing; concentrations can also be used in a population PK analysis, which is reported separately.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Throughout the specification, where compositions are described as including components or materials, it is contemplated that the compositions can also consist essentially of, or consist of, any combination of the recited components or materials, unless described otherwise. Likewise, where methods and uses are described as including particular steps, it is contemplated that the methods and uses can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Throughout the specification, where compositions are described as including components or materials, it is contemplated that the compositions can also consist essentially of, or consist of, any combination of the recited components or materials, unless described otherwise. Likewise, where methods and uses are described as including particular steps, it is contemplated that the methods and uses can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

1. A method for treating major depressive disorder in a human patient in need thereof, comprising administering to said patient a therapeutically effective amount of centanafadine or a pharmaceutically acceptable salt thereof, wherein centanafadine or a pharmaceutically acceptable salt thereof is administered in an extended-release dosage form comprising a plurality of centanafadine beads.

2. The method of claim 1, wherein each of the beads in the plurality of centanafadine beads each comprises a core particle comprising centanafadine or a pharmaceutically acceptable salt thereof, and an excipient, and wherein the plurality of centanafadine beads comprises a mixture of one or more immediate release beads, one or more sustained release beads, and one or more delayed release beads.

3. The method of claim 2, wherein the one or more sustained release beads comprises a sustained release coating comprising one or more materials selected from an alkylcellulose, acrylic acid polymer, a methacrylic acid polymer, an acrylic acid copolymer, a methacrylic acid copolymer, and a cellulose ether.

4. The method of claim 2 or 3, wherein the one or more delayed release beads comprises a delayed release coating comprising one or more materials selected from amylose acetate phthalate, cellulose acetate phthalate, cellulose acetate succinate, cellulose acetate trimellitate, carboxymethyl ethylcellulose, co-polymerized methacrylic acid/methacrylic acid methyl esters, co-polymerized methacrylic acid/methyl methacrylate, co-polymerized methylacrylate/methyl methacrylate/methacrylic acid, hydroxypropyl methylcellulose acetate succinate, hydroxypropyl methylcellulose phthalate, polyvinyl acetate phthalate, styrene maleic acid copolymer, and styrene vinylpyridine copolymer.

5. The method of any one of claim 2 to 4, wherein the mixture of the one or more immediate release beads, one or more sustained release beads, and one or more delayed release beads contain centanafadine or salt thereof at ratio in a range of about 0.1-1:1-20:1-20 parts by weight based on the weight of the centanafadine or salt thereof.

6. The method of any one of claim 1 to 5, wherein administering comprises administering the extended-release dosage form once per day.

7. The method of claim 6, wherein the extended-release dosage form is a capsule.

8. The method of any one of claim 1 to 7, wherein the pharmaceutically acceptable salt of centanafadine is the hydrochloride salt.

9. The method of any one of claim 1 to 8, wherein the amount of the centanafadine or a pharmaceutically acceptable salt thereof administered is in a range of about 10 mg to about 750 mg daily.

10. The method of claim 9, wherein the amount of the centanafadine or a pharmaceutically acceptable salt thereof administered is in a range of about 35 mg to about 400 mg daily.

11. A method for treating major depressive disorder in a patient in need thereof which comprises administering to said patient, a therapeutically effective amount of centanafadine or a pharmaceutically acceptable salt thereof, as adjunct to one or more antidepressants.

12. The method of claim 11, wherein the one or more antidepressants is/are selected from selective serotonin reuptake inhibitors, serotonin-norepinephrine reuptake inhibitors, benzodiazepines, anxiolytics, noradrenergic and specific serotonergic antidepressants, tricyclic antidepressants and monoamine oxidase inhibitors.

13. The method of claim 11, wherein the one or more antidepressants are selected from selective serotonin reuptake inhibitors.

14. The method of claim 13, wherein the selective serotonin reuptake inhibitor comprises escitalopram.

15. The method of any one of claim 11 to 14, wherein centanafadine or a pharmaceutically acceptable salt thereof, is administered in an extended-release dosage form comprising a plurality of centanafadine beads.

16. The method of any one of claim 11 to 15, wherein each of the beads in the plurality of centanafadine beads comprises a core particle comprising centanafadine or a pharmaceutically acceptable salt thereof, and an excipient, and wherein the plurality of centanafadine beads comprises a mixture of one or more immediate release beads, one or more sustained release beads, and one or more delayed release beads.

17. The method of any one of claim 11 to 16, wherein the one or more sustained release beads comprises a sustained release coating comprising one or more materials selected from an alkylcellulose, acrylic acid polymer, a methacrylic acid polymer, an acrylic acid copolymer, a methacrylic acid copolymer, and a cellulose ether.

18. The method of any one of claim 11 to 17, wherein the one or more delayed release beads comprises a delayed release coating comprising one or more materials selected from amylose acetate phthalate, cellulose acetate phthalate, cellulose acetate succinate, cellulose acetate trimellitate, carboxymethyl ethylcellulose, co-polymerized methacrylic acid/methacrylic acid methyl esters, co-polymerized methacrylic acid/methyl methacrylate, co-polymerized methylacrylate/methyl methacrylate/methacrylic acid, hydroxypropyl methylcellulose acetate succinate, hydroxypropyl methylcellulose phthalate, polyvinyl acetate phthalate, styrene maleic acid copolymer, and styrene vinylpyridine copolymer.

19. The method of any one of claim 16 to 18, wherein the mixture of the one or more immediate release beads, one or more sustained release beads, and one or more delayed release beads contain centanafadine or salt thereof at ratio in a range of about 0.1-1:1-20:1-20 parts by weight based on the weight of the centanafadine or salt thereof.

20. The method of any one of claim 11 to 19, wherein the amount of the centanafadine or a pharmaceutically acceptable salt thereof is administered in a range of about 10 mg to about 750 mg daily as adjunct to escitalopram or a pharmaceutically acceptable salt thereof in amount of about 2.5 to about 20 mg daily.

21. The method of claim 20, wherein the amount of the centanafadine or a pharmaceutically acceptable salt thereof administered is in a range of about 35 mg to about 400 mg daily as adjunct to escitalopram or a pharmaceutically acceptable salt thereof in amount of about 10 to about 20 mg daily.

22. A method for treating major depressive disorder in a patient in need thereof which comprises administering to said patient, a therapeutically effective amount of centanafadine or a pharmaceutically acceptable salt thereof, as adjunct to a selective serotonin reuptake inhibitor;
wherein the centanafadine or a pharmaceutically acceptable salt thereof, is administered in an extended-release dosage form comprising a plurality of centanafadine beads;
wherein each of the beads in the plurality of centanafadine beads comprises a core particle comprising centanafadine or a pharmaceutically acceptable salt thereof, and an excipient;
wherein the plurality of centanafadine beads comprises a mixture of one or more immediate release beads, one or more sustained release beads, and one or more delayed release beads;
wherein the mixture of the one or more immediate release beads, one or more sustained release beads, and one or more delayed release beads contain centanafadine or salt thereof at ratio in a range of about 0.1-1:1-20:1-20 parts by weight based on the weight of the centanafadine or salt thereof;
wherein centanafadine or a pharmaceutically acceptable salt thereof is administered in a range of about 35 mg to about 400 mg daily;
wherein the selective serotonin reuptake inhibitor is escitalopram a pharmaceutically acceptable salt thereof; and
wherein the escitalopram or a pharmaceutically acceptable salt thereof is administered in a range of about 10 mg to about 20 mg daily.

23. The method of claim 22, wherein cetanafadine is administered as the hydrochloride salt and escitalopram is administered as the oxalate salt.

24. The method of claim 22 or 23, wherein the mixture of the one or more immediate release beads, one or more sustained release beads, and one or more delayed release beads contain centanafadine or salt thereof at ratio in a range of about 1:3.6:3.6 parts by weight based on the weight of the centanafadine or salt thereof.

25. The method of any one of claim 22 to 24, wherein centanafadine hydrochloride is administered in an amount of 328.4 mg daily and escitalopram oxalate is administered in an amount of 10 mg daily.

26. The method of any one of claim 22 to 24, wherein centanafadine hydrochloride is administered in an amount of 328.8 mg daily and escitalopram oxalate is administered in an amount of 20 mg daily.
